(19) United States
(12) Patent Application Publication
(10) Pub. No.: US 2023/0173018 A1
(43) Pub. Date: Jun. 8, 2023

(54) CASPOFUNGIN DERIVATIVES AND ASSAYS FOR EVALUATING ANTIFUNGAL TREATMENT EFFICACY

(71) Applicant: RAMOT AT TEL AVIV UNIVERSITY LTD., Tel Aviv (IL)

(72) Inventors: Micha FRIDMAN, Tel Aviv (IL); Qais Z JABER, Tel Aviv (IL); Judith BERMAN, Tel Aviv (IL)

(21) Appl. No.: 18/106,009

(22) Filed: Feb. 6, 2023

Related U.S. Application Data
(63) Continuation of application No. PCT/IL2021/050990, filed on Aug. 15, 2021.
(60) Provisional application No. 63/066,407, filed on Aug. 17, 2020.

(51) Int. Cl. A61K 38/12; A61P 31/10; C12Q 1/18

(52) U.S. Cl. CPC A61K 38/12; A61P 31/10; C12Q 1/18

(57) ABSTRACT

Provided herein are caspofungin derivatives having anti-fungal activity and methods of using same for treating fungal infection. Further provided are methods and kits for determining responsiveness to an anti-fungal activity of an echinocandin compound.

### CROSS REFERENCE TO RELATED APPLICATIONS

This application is bypass continuation of PCT Patent Application No. PCT/IL2021/050990 having International filing date of Aug. 15, 2021, which claims the benefit of priority of U.S. Provisional Patent Application No. 63/066,407, filed Aug. 17, 2020, the contents of which are all incorporated herein by reference in their entirety.

### FIELD OF THE INVENTION

Provided herein are caspofungin derivatives having anti-fungal activity and methods of using same for treating fungal infection. Further provided are methods and kits for determining sensitivity to an anti-fungal activity of an echinocandin compound.

### BACKGROUND

Echinocandins are the most recently approved antifungal drugs for clinical use, out of the three main classes of antifungal drugs currently available for treatment of invasive fungal infections. The three echinocandins approved for clinical use by the FDA (caspofungin, micafungin and anidulafungin, approved in 2001, 2005 and 2006, respectively) are considered among the most effective and best-tolerated antifungals in clinical use against Candida species, the most prevalent fungal pathogens of humans in western hospitals. Rezafungin (CD101), a newly developed echinocandin currently undergoing advanced clinical trials, has an extended half-life enabling a single weekly dose.

Although echinocandins are becoming the drugs-of-choice for antifungal treatments, they are not always effective (faller, M. et all, J. Clin. Microbiol. 2011, 49 (2), 624-629, https://doi.org/10.1128/JCM.02120-10; Beyda, N. D. et al., Ann. Pharmacother. 2012, 46 (7-8), 1086-1096, https://doi.org/10.1345/aph.1R020; Dannaoui, E. et al., Emerg. Infect. Dis. 2012, 18 (1), 86-90, https://doi.org/10.3201/eid1801.110556; and Maubon, D. et al., Intensive Care Med. 2014, 40, 1241-1255, https://doi.org/10.1007/s00134-014-3404-7). This may be due their hydrophobicity and differences in the distribution of these drugs at different infection sites. Other possible explanations for efficacy limitations and the continued appearance of echinocandin tolerant and resistant isolates have not been ruled out.

There is an unmet need for effective anti-fungal compounds and for reliable and rapid assays for evaluating responsiveness to antifungal compounds, in order to avoid administration of non-effective treatments.

### SUMMARY

There are provided caspofungin derivatives having anti-fungal activity and use thereof for treating fungal infection. Further provided are a method and a kit for determining responsiveness and resistance to antifungal activity of echinocandins.

The caspofungin derivatives disclosed herein were designed by functionalizing the phenol of 3 S,4S-dihydroxy-L-homotyrosine of the cyclic hexapeptide of caspofungin with a propargyl group. Unexpectedly, this modification, which facilitates click reaction-based conjugation of fluorophores, has been found to serve as a scaffold suitable for obtaining derivatives of caspofungin having anti-fungal activity. Surprisingly, the caspofungin derivatives disclosed herein, namely, compounds/probe 1a, 1 and 2 (see, for example, FIG. 1A), where shown to exert antifungal activity.

Further disclosed herein is a rapid, efficient and reliable assay for identifying yeast cells in test samples, that are sensitive or resistant to the antifungal effect induced by an echinocandin compound. This assay is advantageous in view of the fact that to date, although echinocandins are the preferred antifungal medications, these compounds are not effective in all strains. Thus, the method disclosed herein enables to design an effective antifungal treatment, saving time, costs and pain associated with ineffective treatments. The assay is based on detecting accumulation of the echinocandin candidate in the vacuoles of the tested cells, which was unexpected, in view of the large size of these drugs (MW>1 kDa), thereby suggesting that echinocandins should localize mainly on cell surface while accumulation of the echinocandin candidate in the vacuoles of the tested cells indicates resistance to the antifungal activity exerted thereby.

According to some embodiments there is provided a caspofungin derivative comprising a modified phenol, wherein the caspofungin derivative is having anti-fungal activity.

According to some embodiments, the modified phenol comprises an azide moiety or a propargyl group.

According to some embodiments, the modified phenol comprises a propargyl group. According to some embodiments, the caspofungin derivative is represented by the formula of Compound 1a.

According to some embodiments, the modified phenol comprises an azide moiety. According to some embodiments, the azide moiety comprises 3-azide propylamine. According to some embodiments, the caspofungin derivative is represented by the formula of Compound 1. According to some embodiments, the caspofungin derivative is represented by the formula of Compound 2.

According to some embodiments, the caspofungin derivative is selected from the group consisting of:

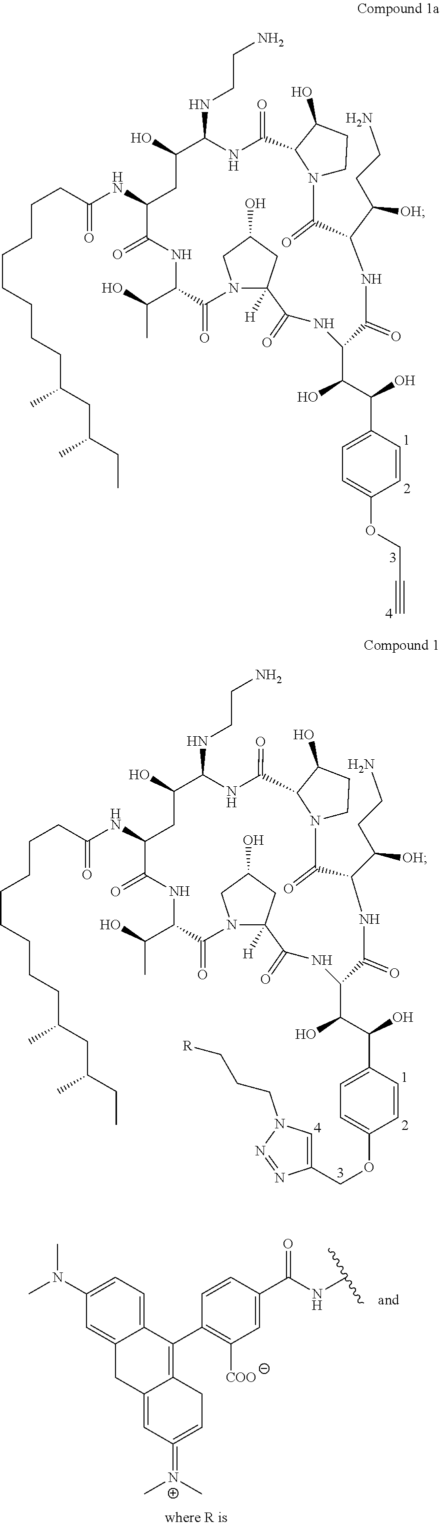

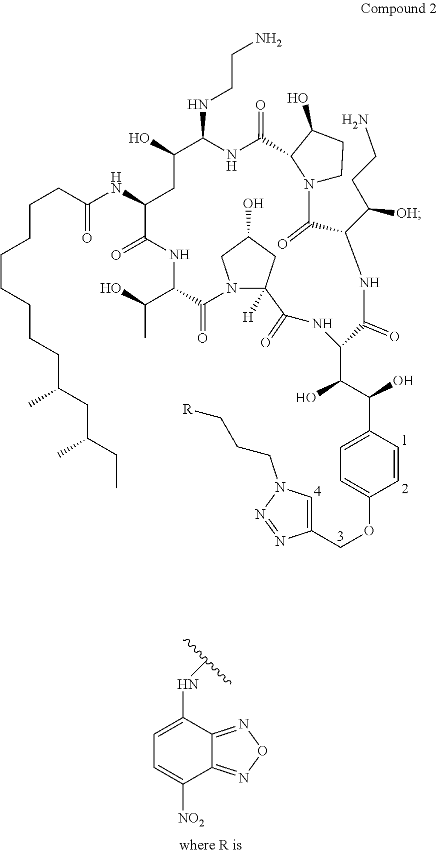

According to some embodiments, there is provided a pharmaceutical composition comprising the caspofungin derivative disclosed herein, and a pharmaceutically acceptable excipient.

According to some embodiments, the pharmaceutical composition disclosed herein is for use in treatment of fungal infection.

According to some embodiments, the fungal infection is an invasive fungal infection.

According to some embodiments, there is provided a method of treating fungal infection in a subject in need thereof, the method comprising administering to the subject in need thereof a pharmaceutical composition comprising the caspofungin derivative disclosed herein.

According to some embodiments, said administering includes topical administration.

According to some embodiments, there is provided a method for determining responsiveness to an anti-fungal activity of an echinocandin compound, the method comprising contacting a yeast cell with an echinocandin compound and determining vacuolar uptake of the echinocandin compound.

According to some embodiments, vacuolar uptake below threshold indicates that said yeast cell is responsive to the antifungal activity of said echinocandin compound.

According to some embodiments, the echinocandin compound comprises a detectable label, and said determining comprises detecting the level of the detectable label.

According to some embodiments, the detectable label comprises fluorophores.

According to some embodiments, the echinocandin compound comprises caspofungin or caspofungin derivative.

According to some embodiments, vacuolar uptake above threshold indicates that said yeast cell is resistant to the antifungal activity of said echinocandin compound.

According to some embodiments, there is provided a method of treating fungal infection in a subject in need thereof, the method comprising
(a) obtaining a sample from a tissue derived from the subject in need thereof;
(b) contacting the sample with echinocandin compound;
(c) determining the vacuolar uptake of the echinocandin compound; and
(d) administering to the subject in need thereof a pharmaceutical composition comprising the echinocandin compound when the vacuolar uptake of the echinocandin compound is below threshold.

According to some embodiments, the echinocandin compound comprises caspofungin or a caspofungin derivative.

According to some embodiments, the echinocandin compound comprises caspofungin.

According to some embodiments, the echinocandin compound comprises Compound 1a. According to some embodiments, the echinocandin compound comprises Compound 1. According to some embodiments, the echinocandin compound comprises Compound 2.

According to some embodiments, the echinocandin compound comprises a detectable label.

According to some embodiments, the echinocandin compound is selected from Compound 1 and Compound 2.

According to some embodiments, the tissue is selected from: mucus secretion, blood, saliva, urine, plasma and epithelial tissue.

According to some embodiments, there is provided a kit for determining responsiveness to an anti-fungal activity of an echinocandin compound, the kit comprising an echinocandin compound, a reference threshold for responsiveness to an anti-fungal activity of the echinocandin compound, and instructions for use.

According to some embodiments, the echinocandin compound comprises a detectable label.

According to some embodiments, the reference threshold comprise a receptacle comprising yeast strain responsive to the anti-fungal activity of the echinocandin compound, thereby a reaction of a test cell or cell culture to the echinocandin compound similar to the reaction of the yeast strain indicates that the test cell or cell culture is responsive to the anti-fungal activity of the echinocandin compound.

According to some embodiments, the reference threshold comprise a receptacle comprising yeast strain resistant to the anti-fungal activity of the echinocandin compound, thereby a reaction of a test cell or cell culture to the echinocandin compound similar to the reaction of the yeast strain indicates that the test cell or cell culture is resistant to the anti-fungal activity of the echinocandin compound.

According to some embodiments, the echinocandin compound comprises caspofungin or a caspofungin derivative. According to some embodiments, the echinocandin compound comprises caspofungin. According to some embodiments, the echinocandin compound comprises Compound 1a. According to some embodiments, the echinocandin compound comprises a detectable label. According to some embodiments, the echinocandin compound is selected from Compound 1 and Compound 2.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIGS. 1A and 1B are a schematic illustrations describing the synthesis of fluorescent caspofungin Compounds 1a, 1 and 2; and the structure of BODIPY-labeled caspofungin probe, respectively.

FIG. 1C is a schematic illustration describing the synthesis of BODIPY-labeled caspofungin probe (CSF-BOD, FIG. 1B) by a site-selective attachment of the BODIPY dye.

FIG. 2 presents the antifungal activity of caspofungin and their semisynthetic derivatives (probes 1a, 1 and 2 and CSF-BOD) on strains related to C. albicans (squares) and C. glabrata families (circles).

FIG. 3A presents time-points of the subcellular distribution of fluorescent caspofungin probe 1 in C. albicans SC5314 cells over 60 min prior to incubation with probes (DIC), following incubation with probe 1 (1 μM), incubation with the vacuole-specific fluorescent dye CellTracker™ Blue CMAC (10 μM,) in phosphate buffered saline (PBS) and images reflecting the combination of the two probes/dyes (Merge). Scale bars, 5 μm.

FIG. 3B presents time-points of the subcellular distribution of fluorescent caspofungin probe 1 in C. albicans (SN152, ATCC 24433) and C. glabrata (ATCC 2001, ATCC 66032) cells over 60 min prior to incubation with probes (DIC), following incubation with probe 1 (1 μM), incubation with the vacuole-specific fluorescent dye CellTracker™ Blue CMAC (10 μM,) in phosphate buffered saline (PBS) and images reflecting the combination of the two probes/dyes (Merge). Scale bars, 5 μm.

FIG. 3C presents time-points of the subcellular distribution of fluorescent caspofungin probe 1 in C. albicans CG72 cells expressing Ypt72-GFP over 60 min prior to incubation with probes (DIC), following incubation with probe 1 (1 μM), in phosphate buffered saline (PBS) and images reflecting the combination of the two probes/dyes (Merge). Scale bars, 5 μm.

FIG. 3D presents time-points of the subcellular distribution of fluorescent caspofungin probe 2 in C. albicans SC5314 and C. glabrata ATCC 2001 cells. Cells were incubated with probe 2 (1 μM, green) and with CellTracker™ Blue CMAC (10 μM, blue) in PBS for 60 min. Merged staining of Probe 2 and CMAC is shown on the right panels. Scale bars, 5 μm.

FIG. 3E presents subcellular distribution of fluorescent caspofungin CSF-BOD in C. albicans SC5314 and C. glabrata ATCC 2001 cells. Cells were incubated with CSF-BOD (1 μM) and with CellTracker™ Blue CMAC (10 μM) in PBS for 60 min. Merged staining of CSF-BOD and CMAC is shown on the right panels. Scale bars, 5 μm.

FIG. 3F presents subcellular distribution of fluorescent caspofungin CSF-BOD in Candida cells SC 5314 and ATCC 2001, treated with BODIPY-methyl ester (1 μM, green) in PBS for 60 min.

FIG. 3G presents C. albicans CG72 cells expressing Ypt72-GFP incubated with free dye 5-TMR-azide (1 μm), where merged staining of 5-TMR-azide and Ypt72-GFP is shown on the right panels. Cells were incubated in PBS for 60 min. Scale bars, 5 μm.

FIG. 3H presents C. albicans (SC5314, SN152, ATCC 24433) and C. glabrata (ATCC 2001, ATCC 66032) cells incubated with free dye 5-TMR-azide (1 μm) and with CellTracker™ Blue CMAC (10 μM) where merged staining of 5-TMR-azide and CMAC is shown on the right panels. Cells were incubated in PBS for 60 min. Scale bars, 5 μm.

FIG. 3I presents C. albicans SC5314 and C. glabrata ATCC 2001 cells incubated with free dye NBD-azide (1 μm) and with CellTracker™ Blue CMAC (10 μM) where merged staining of NBD-azide and CMAC is shown on the right panels. Cells were incubated in PBS for 60 min. Scale bars, 5 μm.

FIGS. 4A and 4B present the subcellular distribution of fluorescent caspofungin probe 1 and 2 in C. albicans CG72 cells expressing Ypt72-GFP following incubation with probe 1 (1 μM) for 60 min in PBS (4A) and in C. albicans SC5314 cells following incubation with probe 2 (1 μM) and with FM4-64 (1 μg/mL) for 60 min in PBS. Merged staining is shown on the right panels. Scale bars, 5 μm.

FIG. 4C presents subcellular distribution of FM4-64 (1 μM/mL) in C. albicans SC5314 cells over a 60-min time course. Merged staining is shown on the right panels. Cells were incubated with FM4-64 and with the vacuole-specific fluorescent dye CellTracker™ Blue CMAC (10 μM) in PBS. Scale bars, 5 μm.

FIG. 5A presents flow cytometry analysis of the uptake of probe 1 over time (in arbitrary units, A.U.). Data are presented as means±standard deviations (SD; error bars). Significance was determined by an unpaired t test (ns indicates not significant with P >0.05). The integrated densities (in relative fluorescent units, RFU) per cell were determined from microscopic images with ImageJ. Data are presented as means±SD (˜3000 cells).

FIG. 5B presents microscopy images of C. albicans SC5314 cells treated with probe 1 for 15 minutes. The DIC images were processed with a Frangi vesselness filter using ImageJ to show the cell borders (left images), merged images of the fluorescent and the DIC channels (right images). Scale bars, 5 μm.

FIG. 6A presents the effect of endocytosis inhibitors on fluorescent caspofungin probe 1 uptake in C. albicans SC5314 and SN152 cells, with or without pre-incubation with 8 μg/mL of endocytosis inhibitors TFP or CGS 12066B, in the presence of 1 μM of probe 1 in PBS. Data are presented as mean±SD (error bars), significance was determined by an unpaired t test (*P<0.05, **P<0.01).

FIG. 6B presents the effect of endocytosis inhibitors on fluorescent caspofungin probe 1 uptake in C. albicans SC5314 and SN152 cells, with or without addition of caspofungin (1 μM, 10 μM) or fluconazole (FLC) (10 μM). Data are presented as mean±SD (error bars), significance was determined by an unpaired t test (*P<0.05, **P<0.01, *** P≤0.001; ns indicates not significant with P>0.05).

FIGS. 6C-6F present the effect of TFP and CGS 12066B on the growth of the following Candida cells: C. albicans SC5314 and C. albicans SN152 (C, D respectively), and the effect of TFP on the growth of C. albicans SC5314 and C. albicans SN152 (E, F respectively).

FIG. 7A presents the dynamics of probe 1 localization and cell death in C. albicans SC5314 cells as a function of the incubation medium (PBS vs YPAD) in cells treated with probe 1 (1 μM) in PBS or YPAD and untreated cells (no probe 1 addition; DIC). Scale bars, 5 μm.

FIG. 7B presents the dynamics of probe 1 localization and cell death in C. albicans SC5314 cells as a function of the incubation medium (PBS vs YPAD) in cells treated with caspofungin (1 μM) and stained with PI (20 μM) in PBS or YPAD and untreated cells (PI staining with no caspofungin addition). The negative control of untreated cells identical in PBS and YPAD. Scale bars, 5 μm.

FIG. 8A presents subcellular distribution of probe 1 on caspofungin resistant strains carrying FKS mutations, C. albicans (strains no. 2 and 3, Table 1) and C. glabrata (strains no. 17 and 27, Table 1), following incubation with the probe 1 (1 μm, red) for 60 min in PBS. Scale bars, 5 μm.

FIG. 8B presents comparison of probe 1 uptake by parental and corresponding mutant C. albicans (Strains no. 1-4, Table 1) and C. glabrata (Strains no. 16-38, Table 1)—strains carrying FKS mutations, where the intracellular fluorescence of parental strains (deep shade) and their corresponding mutant strains (lighter shades) were analyzed by flow cytometry after 15 minutes of incubation with probe 1 (1 μM). Data are presented as mean±SD (error bar). Significance was determined by an unpaired t test (*P<0.05, **P<0.01, ***P<0.001, and ns=not significant=P>0.05).

FIG. 8C presents comparison of the uptake of caspofungin probe 1 by parental and corresponding mutant C. albicans (Strains no. 1-4, Table 1) and C. glabrata (Strains no. 16-38, Table 1) strains carrying FKS mutations. Intracellular fluorescence of parental strains (dark columns) and their corresponding mutant strains (light coloured columns) were analyzed by flow cytometry after 30, 45, and 60 minutes of incubation with probe 1 (1 μM). Data are presented as mean±SD (error bars).

FIG. 8D presents comparison of the uptake of caspofungin probe 2 by parental and corresponding mutant C. albicans (Strains no. 1-4, Table 1) and C. glabrata (Strains no. 18-20 and Strains no. 31-33, Table 1) strains carrying FKS mutations. Intracellular fluorescence of parental strains (dark columns) and their corresponding mutant strains (light coloured columns) were analyzed by flow cytometry after 15, 30, and 45 minutes of incubation with probe 2 (1 μM). Data are presented as mean±SD (error bars).

FIG. 8E presents comparison of the uptake of caspofungin probe CSF-BOD by parental and corresponding mutant C. albicans (Strains no. 1-4, Table 1) and C. glabrata (Strains no. 18-20 and Strains no. 31-33, Table 1) strains carrying FKS mutations. Intracellular fluorescence of parental strains (dark columns) and their corresponding mutant strains (light coloured columns) were analyzed by flow cytometry after 15, 30, and 45 minutes of incubation with probe CSF-BOD (1 μM). Data are presented as mean±SD (error bars).

FIGS. 9A and 9B present caspofungin probe 1 uptake following 15 minutes of incubation, comparing caspofungin-responsive strains (C. albicans and C. glabrata MIC≤0.25 μg/mL and 0.12 μg/mL, respectively) and caspofungin-resistant strains (C. albicans and C. glabrata MIC ≥1 μg/mL and 0.5 μg/mL, respectively), where the horizontal dashed lines indicated the suggested cutoff values defined for probe 1 (1 μM) uptake based upon receiver operating characteristics (ROC) curves using SPSS software. Significance of the difference between the uptake of probe 1 by the group of resistant strains and the group of responsive strains was determined by an unpaired t test (*P<0.05; ****P<0.0001).

FIG. 10A represents comparison of the level of chitin in parental strains (deep shade) and corresponding mutant C. albicans (Strains 1-4, Table 1; lighter shade) and C. glabrata (Strains 18-20, Table 1; lighter shade), as analyzed by flow cytometry after 30 minutes of incubation with chitin-specific dye calcofluor white—CFW (25 μg/mL). Data are presented as mean±SD (error bar). Significance was determined by an unpaired t test (*P<0.05, **P<0.01, ****P<0.001, and ns=not significant=P>0.05).

FIG. 10B represents the effect of Ca⁺² on the chitin levels in C. albicans SC5314 and C. albicans SN152 cells. Data are presented as mean±SD (error bar). Significance was determined by an unpaired t test (*P<0.05, **P<0.01, ****P<0.001, and ns=not significant=P>0.05).

FIG. 10C represents the effect of Ca⁺² on probe 1 uptake in C. albicans SC5314 and C. albicans SN152 cells. Data are presented as mean±SD (error bar). Significance was determined by an unpaired t test (*P<0.05, **P<0.01, ****P<0.001, and ns=not significant=P>0.05).

### DETAILED DESCRIPTION

The principles uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

In the description and claims of the application, the words “include” and “have”, and forms thereof, are not limited to members in a list with which the words may be associated.

In some embodiments, there is provided a caspofungin derivative comprising a modified phenol wherein the caspofungin derivative is having anti-fungal activity.

Caspofungin is also known as ((4R,5S)-5-[(2-Aminoethyl)amino]-N2-(10,12-dimethyltetradecanoyl)-4-hydroxy-L-ornithyl-L-threonyl-trans-4-hydroxy-L-prolyl-(S)-4-hydroxy-4-(p-hydroxyphenyl)-L-threonyl-threo-3-hydroxy-L-ornithyl-trans-3-hydroxy-L-proline cyclic (6→1)-peptide. Thus, the modified phenol is the phenol of 3 S,4S-dihydroxy-L-homotyrosine of the cyclic hexapeptide of caspofungin. Modification of the phenol facilitates conjunction of a detectable label.

Caspofungin belongs to the family of echinocandins, which are the only class of clinically approved antifungal drugs that act by inhibiting β-(1→3)-glucan synthase (GS), a membrane-bound protein complex essential for fungal cell-wall biosynthesis. Importantly, GS is present in fungi, but not in animals, which may explain the exceptional safety profile of echinocandins. Echinocandins are semisynthetic drugs, developed from fermentation metabolites, are composed of different hexapeptide scaffolds attached to an N-linked lipid chain that has been modified chemically to optimize pharmacokinetics and pharmacodynamics. Echinocandins are the most recently approved class of clinical antifungal drugs used for treatment of invasive fungal infections.

Fks1p, an essential component of the GS complex, is an approximately 200-kDa protein composed of 16 membrane-spanning domains and encoded by the FKS1 gene. Fks1p is the catalytic subunit that forms the glyosidic linkage in the β-(1→3)-D-glucan polymer, based upon photo-affinity experiments with UDP-D-glucose. Resistance to echinocandins has been associated with point mutation hotspots, with most hotspot mutations conferring resistance to all three echinocandins in clinical use. These Fks1 hotspot regions reside in predicted extracellular domains of the protein that are thought to bind the echinocandins, which act as non-competitive inhibitors of the GS complex.

GS has been implicated as a target for echinocandins by cell-free GS assays showing echinocandin-mediated inhibition of fungal glucan polymer formation from UDP-[¹⁴C]-D-glucose. Genetic experiments support this conclusion: several point-mutation hotspot regions in the GS complex were associated with reduced sensitivity to echinocandin.

Intuitively, echinocandins are expected to localize mainly to the cell surface because of their large size (MW >1 kDa) and membrane anchoring lipid segment. Furthermore, the extracellular orientation of the GS binding site obviates the need for the drug to enter cells to be efficacious. Yet, ³H-labeled-caspofungin readily accumulated in the cytoplasm of C. albicans cells. This uptake of the drug is thought to occur via a high-affinity transporter at a concentration of ≥1 μg/mL along with non-selective diffusion across the plasma membrane at higher drug concentrations. A study providing low resolution images of a BODIPY-labeled caspofungin probe, suggested that it localized to germ tubes along with possible vesicle involvement in C. albicans. (Pratt, A. et al., Med. Mycol. 2013, 51 (1), 103-107; https://doi.org/10.3109/13693786.2012.685767). This probe was later used in an investigation of the reason for the inherent resistance of the fungal pathogen Cryptococcus neoformans to caspofungin (Huang, W. et al., MBio 2016, 7 (3); https://doi.org/10.1128/mBio.00478-16). It was demonstrated that the BODIPY-labeled caspofungin probe non-specifically stained cells of a C. neoformans mutant with a damaged plasma membrane while it was barely detectable in the wild-type parent.

Thus, according to some embodiments, there is provided a caspofungin derivative comprising a modified phenol, wherein the modified phenol is the phenol of 3 S,4S-dihydroxy-L-homotyrosine of the cyclic hexapeptide of caspofungin.

According to some embodiments, the caspofungin derivative includes a modified phenol to facilitate conjunction of a detectable label thereto. Surprisingly, the caspofungin derivative is having anti-fungal activity.

According to some embodiments, the modified phenol comprises an azide moiety or a propargyl group. In some embodiments, the modified phenol includes an azide moiety. According to some embodiments, the azide moiety comprises 3-azide propylamine.

According to some embodiments, the modified phenol includes a propargyl group According to some embodiments the caspofungin derivative is having the following structure, also termed herein, Compound 1a or probe 1a:

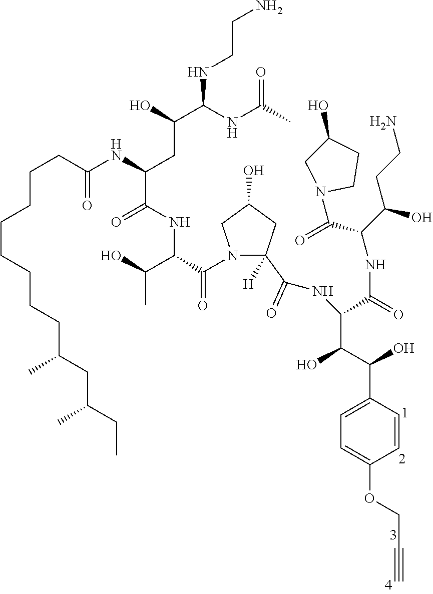

According to some embodiments the caspofungin derivative comprises a detectable label. In some embodiments, the detectable label include fluorophores.

The terms “compound” and “probe” as used herein are interchangeable.

Fluorescent microscopy techniques are broadly applied tools for studying biological processes in living cells. Thus, according to some embodiments, the caspofungin derivative is a fluorescent caspofungin derivative.

According to some embodiments, the caspofungin derivative is having the following structure:

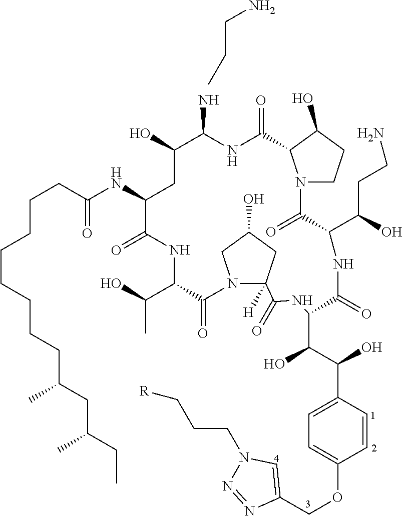

wherein R is an azide moiety.

According to some embodiments, the azide moiety includes 3-azide propylamine. According to some embodiments, the azide moiety further includes rhodamine. According to some embodiments, the azide moiety further includes nitrobenzoxadiazole.

According to some embodiments, the caspofungin derivative is having the following structure, also termed herein, Compound 1 or probe 1:

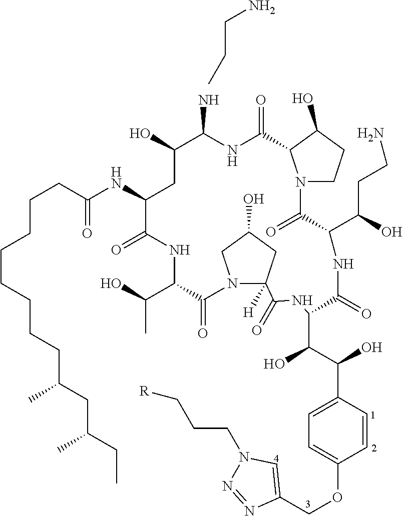

wherein R is:

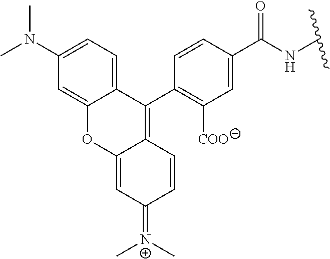

According to some embodiments, the caspofungin derivative is having the following structure, also termed herein, Compound 2 or probe 2:

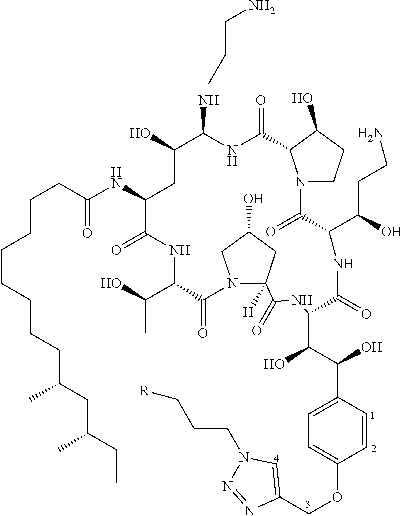

wherein R is:

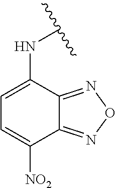 [SMILES: *Nc1ccc([N+](=O)[O-])c2nonc12]

Through a facile four-step synthesis, fluorescently labeled probes of caspofungin were synthesized. These probes enable live-cell imaging by microscopy and flow cytometry to characterize the organellar sites of drug localization and the degree of drug uptake across a panel of Candida isolates, as exemplified herein with exemplary derivatives 1a, 1 and 2. Advantageously, these compounds were found to accumulates in vacuoles within minutes, likely via endocytosis. Using live cell imaging, it has been found that the cellular uptake of the fluorescent drug was energy dependent. Notably, time-dependent subcellular distribution of the fluorescent drug indicated that echinocandins cause more cell death under conditions that promote rapid yeast cell growth; when cells kept in glucose-free buffered water, the drug accumulates and remains in the vacuole. Thus, echinocandins are more effective against metabolically active and dividing yeast cells and are less effective in slow-dividing and/or dormant yeast cells.

Thus, there is provided, a method for treating fungal infection in a subject in need thereof, the method includes administering to the subject an effective amount of a pharmaceutical composition comprising the caspofungin derivatives disclosed herein.

In some embodiments, administering includes any one or more of intravenous injection, intramuscular injection, intraperitoneal injection, infusion, subcutaneous injection, transdermal, aerosol, rectal, vaginal, topical, oral or inhaled delivery.

The term “effective amount” as used herein includes any amount and/or concentration, which provides an effective therapy of the fungal infection. The effective amount may be used once, or a plurality of time, daily, weekly, monthly, under any treatment regimen that provided an effective healing of the fungal infection.

According to some embodiments, treatment comprises alleviation of the fungal infection, inhibition of progression of the fungal infection and cure of the fungal infection. In some embodiments, treatment comprises prevention of a fungal infection.

Sterile injectable solutions can be prepared by incorporating the active echinocandin compound in the required amount in a selected solvent, as required, followed by filtered sterilization. Generally, dispersions are prepared by incorporating the active echinocandin compound into a sterile vehicle, which contains a basic dispersion medium and other ingredients if required. In the case of sterile powders for the preparation of sterile injectable solutions, the methods of preparation may include vacuum drying and freeze-drying which may yield a powder of the active echinocandin ingredient.

Oral compositions generally include an inert diluent or an edible carrier. For the purpose of oral therapeutic administration, the active echinocandin compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the echinocandin compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

According to some embodiments, there is provided a method for determining sensitivity to an anti-fungal activity of an echinocandin compound, the method comprising contacting a yeast cell with an echinocandin compound and determining the presence of the echinocandin compound in the vacuole of said yeast cell.

As disclosed herein and exemplified below, quantification of echinocandin vacuole uptake rapidly and accurately indicates whether a cell is echinocandin-resistant or echinocandin-sensitive. Hence, methods disclosed herein offer a new and useful tool for predicting treatment efficacy of fungal infections, including, invasive life-threatening fungal infections. Enhanced vacuolar uptake of echinocandin characterizes echinocandin-resistant pathogenic yeast while reduced vacuolar uptake of the echinocandin characterizes echinocandin-sensitive pathogenic yeast. Accordingly, the methods disclosed herein are particularly useful for devising optimal drug-treatments for fungal infections.

According to some embodiments, vacuolar uptake below threshold indicates that said yeast cell is responsive to the antifungal activity of said echinocandin compound and vacuolar uptake above threshold indicates that said yeast cell is resistant to the antifungal activity of said echinocandin compound.

The term “threshold” as used herein refers to a numerical value which can be used as a specific and sensitive cutoff distinguishing between resistance and responsiveness (sensitivity) to antifungal activity of a given echinocandin. The threshold may be a statistical value, calculated from a plurality of values reflecting resistance and responsiveness to a given echinocandin as measured in cells, yeast strains and the like, having known tendency resistance and responsiveness) to the given echinocandin.

The method for determining the efficacy of echinocandin antifungal activity, as disclosed herein is based on analyzing whether vacuolar uptake of a given echinocandin corresponds to responsiveness to the echinocandin or resistance thereto. Thus, in some embodiments, the method comprises comparing a measured vacuolar uptake for an echinocandin compound is a test sample, to a threshold value, or a plurality of threshold values corresponding to vacuolar uptake of the echinocandin compound in cells that are resistant to said echinocandin compound. In some embodiments, the method comprises comparing a measured vacuolar uptake for an echinocandin compound in a test sample, to a threshold value, or a plurality of threshold values corresponding to vacuolar uptake of the echinocandin compound in cells that are responsive/sensitive to said echinocandin compound. In some embodiments, the method comprises comparing a measured vacuolar uptake for an echinocandin compound in a test sample, to a scale of threshold values ranging from threshold values corresponding to vacuolar uptake of the echinocandin compound in cells that are sensitive to said echinocandin compound to threshold values corresponding to vacuolar uptake of the echinocandin compound in cells that are resistant to said echinocandin compound.

According to some embodiments, the comparison yields a score (probability score) reflecting the likelihood that the measured vacuolar uptake corresponds to resistance to the echinocandin compound or the likelihood that the measured vacuolar uptake corresponds to sensitivity to the echinocandin compound. The better approximation of the measure vacuolar uptake to a particular reference/threshold, the higher the score (probability score) and accordingly the likelihood that the measure vacuolar uptake corresponds to the specific tendency (responsiveness/sensitivity or resistance). In some embodiments, the probability score is based on the relative position of the measured vacuolar uptake within the distribution of the particular threshold values.

According to some embodiments, there is provided a method of treating fungal infection in a subject in need thereof, the method comprising
a. obtaining a sample from a tissue derived from the subject in need thereof;
b. contacting the sample with echinocandin compound;
c. determining the vacuolar uptake of the echinocandin compound; and
d. administering to the subject in need thereof a pharmaceutical composition comprising the echinocandin compound when the vacuolar uptake of the echinocandin compound is below threshold.

According to some embodiments, the tissue includes, but is not limited to, any one or more of blood, plasma, saliva and urine.

According to some embodiments, the method comprises administering to the subject in need thereof a pharmaceutical composition comprising the antifungal compound other than the echinocandin compound when the vacuolar uptake of the echinocandin compound is above threshold.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1: Design and Synthesis of Fluorescent Caspofungin Probes

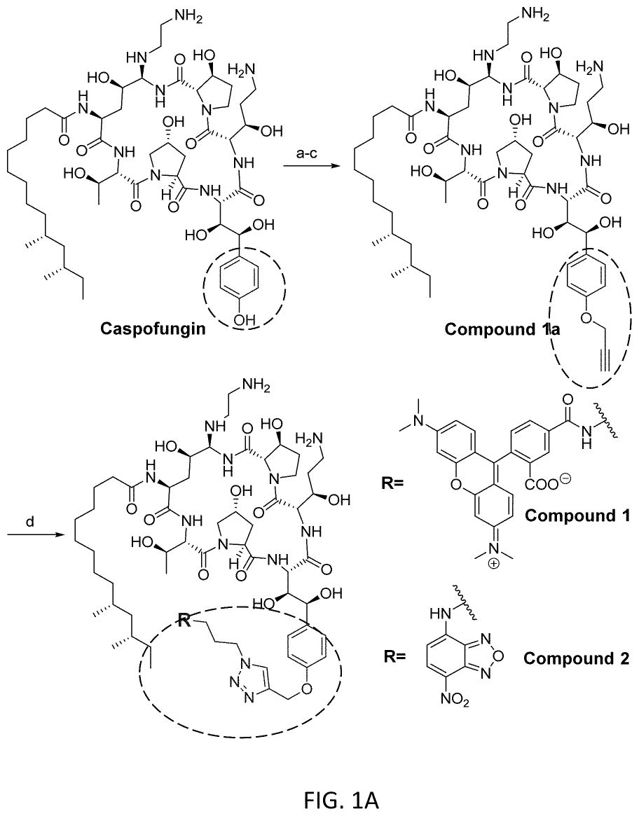

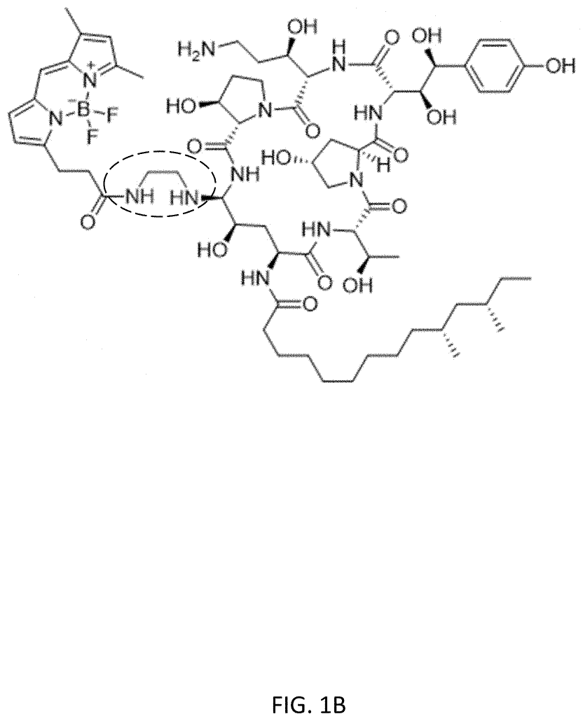

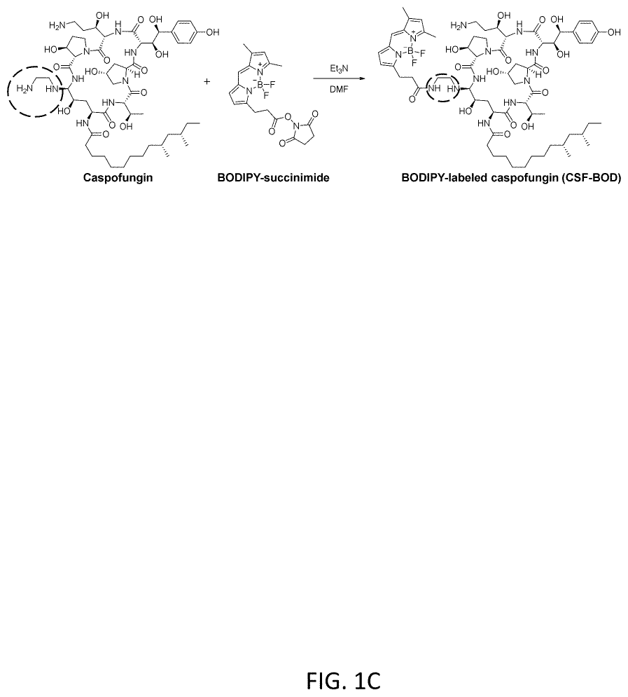

The design of fluorescently labeled caspofungin probes was based on specific functionalization of the phenol of 3S,4S-dihydroxy-L-homotyrosine of the cyclic hexapeptide of caspofungin, by a propargyl group, to facilitate click reaction-based conjugation of different fluorophores as illustrated in FIG. 1. The phenol group of caspofungin (FIG. 1; encircled) was chosen for functionalization, rather than one of the three amines of the cyclic peptide, to avoid reducing the overall positive charge of caspofungin under physiological conditions, which may affect antifungal efficacy and/or solubility.

The synthesis of propargyl-functionalized caspofungin intermediate 1a was accomplished in three steps with an overall isolated yield of 45% (FIG. 1; encircled).

Briefly, the three amine residues of caspofungin were protected with tert-butyloxycarbonyl (BOC) carbamates followed by selective etherification of the phenol with propargyl bromide under basic conditions. Notably, an attempt to remove the BOC carbamates in neat TFA resulted in a mixture of different products with the molecular weight of the desired product (1,131.38 g/mole), presumably, due to isomerization of chiral centers under these acidic conditions. Rapid removal of the BOC groups using hydrogen chloride in aqueous isopropanol afforded the synthesis of Compound 1a as a single product in 90% isolated yield (FIG. 1). The structure of Compound 1a was confirmed by a NOESY NMR that showed strong correlation between the aromatic hydrogens of the 3S,4S-dihydroxy-L-homotyrosine and the protons of the propargyl ether (not shown). Finally, azide functionalized tetramethylrhodamine (TMR) and nitrobenzoxadiazole (NBD) dyes (FIG. 1A) were coupled with Compound 1a under the click reaction conditions to produce fluorescent caspofungin probes 1 (61% isolated yield; absorption-550 nm and emmision-585 nm) and 2 (68% isolated yield; absorption-470 nm and emmision-540 nm), respectively. Overall, the four-step synthetic sequence yielded fluorescent caspofungin probes 1 and 2 in 27% and 30% isolated yields, respectively. The propargyl-functionalized caspofungin 1a that was generated in three steps from the parent drug offers a useful intermediate that can be further modified to generate a large diversity of novel caspofungin derivatives.

Thus, the outline of the synthesis of fluorescent caspofungin probes 1 and 2 included the following steps: a) Boc₂O, dioxane/H₂O, ambient temperature, 48 h, 83%; b) Propargyl bromide in toluene, Cs₂CO₃, DMF, ambient temperature, 14 h, 60%; c) 37% HCl in H₂O/isopropanol (⅓), 2 h, ambient temperature, 90%; d) Azide functionalized fluorescent dye, CuSO₄·5H₂O, sodium ascorbate, DMF, ambient temperature (4 h, 61%) for compound 1 and (3 h, 68%) for compound 2. The phenol group of 3S,4S-dihydroxy-L-homotyrosine amino acid that was modified is encircled in FIG. 1A.

A BODIPY-labeled caspofungin probe (CSF-BOD, FIG. 1B) was synthesized according to the previously reported general procedure (Pratt et al., ibid). This fluorescent probe was generated by a site-selective attachment of the BODIPY dye (FIG. 1C) to the primary amine of the ethylenediamine functionality of the drug via an amide bond.

BODIPY-labeled caspofungin probe (CSF-BOD) was prepared as previously reported⁵ with the following changes: Caspofungin diacetate (100 mg, 0.08 mmol, 2 eq) and triethylamine (25 μL, 2 eq) were dissolved in DMF (4 mL) and treated with BODIPY-succinimide (16 mg, 0.04 mmol, 1 eq), synthesized following a previously reported procedure.⁶ The mixture of the reaction was stirred at ambient temperature for 2 h. Reaction progress was monitored by ESI-MS following the formation of CSF-BOD ([M+H]⁺, m/z 1366.67). Upon completion, the solvent was removed under vacuum. The residue was purified by preparative RP-HPLC (mobile phase: Acetonitrile in H₂O (containing 0.1% TFA), gradient from 10% to 90%; flow rate: 15 mL/min) to afford CSF-BOD (37 mg, 68%) as an orange solid.

Importantly, of the three fluorescent dyes used for the preparation of the caspofungin probes, NBD fluorescence is pH and environment sensitive, whereas TMR and BODIPY are largely unaffected by pH. However, the photostability of the TMR proved to be much higher than that of BODIPY. TMR-labeled caspofungin compound 1 was therefore used as the main probe in this study.

### Example 2. Fluorescent Caspofungin Probes Retain Antifungal Activity

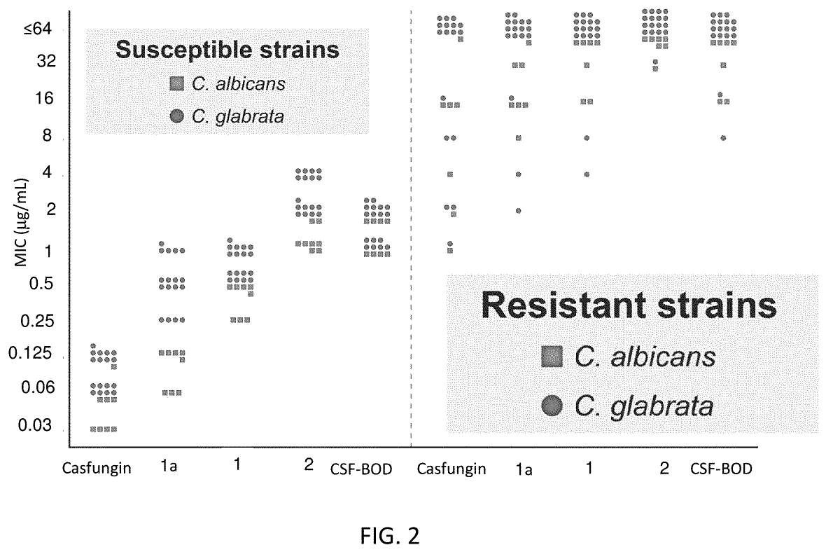

The antifungal activities of compounds 1a, 1 and 2 (collectively: caspofungin derivatives) were compared to that of caspofungin and CSF-BOD using a panel of 49 C. albicans and C. glabrata strains (Tables 1 and 2). The panel included ATCC strains and clinical isolates as well a collection of caspofungin-responsive strains and their corresponding isogenic caspofungin-resistant derivatives, constructed by introducing point mutations within and near the defined hotspots in the FKS1 and/or FKS2 genes of the GS complex. Minimal inhibitory concentration (MIC) values for all strains were determined using the broth double dilution method and are summarized in FIG. 2 and Table 2. MIC values were determined using the broth double-dilution method. The clinical breakpoints (threshold) for caspofungin-resistance in C. albicans and C. glabrata are 1 μg/mL and 0.5 μg/mL, respectively.

TABLE 1
Strains Information.

| | | | Isogenic parental | | |
|---|---|---|---|---|---|
| # | Species | Strain Name | strain | Genotype | Source |
| 1. | C. albicans | SC5314 | WT | | David Perlin⁷ |
| 2. | C. albicans | DPL1015 | SC5314 | FKS1 mutant HS1- | David Perlin⁸ |
| | | (DP-A15) | | S645S/P | |
| 3. | C. albicans | DPL1016 | SC5314 | FKS1 mutant HS1- | David Perlin⁸ |
| | | (DP-A15-10) | | S645P | |
| 4. | C. albicans | T-2068 | SC5314 | FKS1 HS1-F641S | David Perlin⁸ |
| | | (DP-C42) | | mutation | |
| 5. | C. albicans | SN152 | — | | Susan Lindquist⁹ |
| 6. | C. albicans | ATCC 24433 | — | | ATCC |
| 7. | C. albicans | CG72 | CAM | pKE1:GFP-YPT72 | Glen E palmer¹⁰ |
| 8. | C. albicans | SN95 | RM1000 | | Sandy Johnson¹¹ |
| 9. | C. albicans | BWP17 | RM1000 | | Aaron Mitchell¹² |
| 10. | C. albicans | CAF2-1 | SC5314 | ura3: imm434/UR43 | Chantal Fradin¹³ |
| 11. | C. albicans | ATCC 10231 | — | | Susan Lindquist |
| 12. | C. albicans | T-2074 | clinical isolate | FKS1 HS1-P649H | David Perlin¹⁴ |
| | | (DP-122) | | mutation | |
| 13. | C. albicans | T-2076 | clinical isolate | FKS1 HS1-S645F, | David Perlin¹⁴ |
| | | (DP-194) | | HS2-R1361R/H | |
| | | | | mutation | |
| 14. | C. albicans | T-2069 | clinical isolate | FKS1 HS1-S645F | David Perlin¹⁴ |
| | | (DP-85) | | mutation | |
| 15. | C. albicans | T-2077 | clinical isolate | FKS1 HS1-S645P | David Perlin¹⁴ |
| | | (DP-205) | | mutation | |
| 16. | C. glabrata | ATCC 90030 | WT | | David Perlin |
| 17. | C. glabrata | DPL1086 | ATCC 90030 | FKS1 mutant HS1- | David Perlin¹⁵ |
| | | | | S629P | |
| 18. | C. glabrata | CST109 | | | Toni Gabaldon¹⁶ |
| 19. | C. glabrata | TGL00054 | CST109 | FKS2 HS1-F659- | Toni Gabaldon |
| | | | | L664R, HS2-R1378L | |
| 20. | C. glabrata | TGL00253 | CST109 | FKS2 HS1-F659- | Toni Gabaldon |
| | | | | HS2-R1378S | |
| 21. | C. glabrata | CST34 | | | Toni Gabaldon¹⁶ |
| 22. | C. glabrata | TGL00056 | CST34 | FKS1 E621K, FKS2 | Toni Gabaldon |
| | | | | HS1-F659-D666Y, | |
| 23. | C. glabrata | TGL00107 | CST34 | FKS1 HS1-S629P- | Toni Gabaldon |
| | | | | P633Q | |
| 24. | C. glabrata | TGL00256 | CST34 | FKS1 HS1-D632G, | Toni Gabaldon |
| 25. | C. glabrata | TGL00258 | CST34 | FKS2 HS1-F659L- | Toni Gabaldon |
| | | | | D666Y | |
| | | | | FKS1 HS1-S629P | |
| 26. | C. glabrata | CST78 | CST78 | FKS1 HS1-L628I- | Toni Gabaldon¹⁶ |
| 27. | C. glabrata | TGL00065 | | | Toni Gabaldon |
| 28. | C. glabrata | TGL00263 | CST78 | D632N | Toni Gabaldon |
| | | | | FKS2 HS1-F659S | |
| 29. | C. glabrata | TGL00264 | CST78 | FKS2 HS1-R665G, | Toni Gabaldon |
| 30. | C. glabrata | TGL00265 | CST78 | FKS2 HS2-R1378C- | Toni Gabaldon |
| | | | | L1381F | |
| | | | | FKS1 HS1W611*, | |
| 31. | C. glabrata | EF1620 | EF1620 | FKS2 HS1-F659Y | Toni Gabaldon¹⁶ |
| 32. | C. glabrata | TGL00275 | | FKS2 HS1-L662F- | Toni Gabaldon |
| 33. | C. glabrata | TGL00277 | EF1620 | D666N | Toni Gabaldon |
| | | | | FKS1 HS1-F625Y- | |
| 34. | C. glabrata | BG2 | BG2 | D632E | Toni Gabaldon¹⁶ |
| 35. | C. glabrata | TGL00091 | | FKS2 HS1-A651V- | Toni Gabaldon |
| 36. | C. glabrata | TGL00291 | BG2 | S663P | Toni Gabaldon |
| | | | | FKS2 HS1-S654Y- | |
| 37. | C. glabrata | TGL00293 | BG2 | F659- | Toni Gabaldon |
| | | | | FKS2 HS1-A651V- | |
| 38. | C. glabrata | TGL00294 | BG2 | F659- | Toni Gabaldon |
| | | | | FKS2 HS1-L662W, | |
| 39. | C. glabrata | ATCC 2001 | — | HS2-R1378C | Cecile Fairhead¹⁷ |
| 40. | C. glabrata | ATCC 66032 | — | | ATCC |
| 41. | C. glabrata | ATCC 15126 | — | | Ronen Ben Ami¹⁸ |
| 42. | C. glabrata | T-190 (11-304) | clinical isolate | | Ronen Ben Ami¹⁸ |
| 43. | C. glabrata | T-191 (11-331) | clinical isolate | | Ronen Ben Ami¹⁸ |
| 44. | C. glabrata | T-192 (1775) | clinical isolate | | Ronen Ben Ami¹⁸ |
| 45. | C. glabrata | T-193 (11-282) | clinical isolate | | Ronen Ben Ami¹⁸ |
| 46. | C. glabrata | T-982 (11-181) | clinical isolate | | Ronen Ben Ami¹⁸ |
| 47. | C. glabrata | T-983 (11-079) | clinical isolate | | Ronen Ben Ami¹⁸ |
| 48. | C. glabrata | T-984 (11-078) | clinical isolate | | Ronen Ben Ami¹⁸ |
| 49. | C. glabrata | T-985 (11-076) | clinical isolate | | Ronen Ben Ami¹⁸ |

TABLE 2
Minimal Inhibitory Concentration (MIC) values of caspofungin and
semisynthetic derivatives (parental strains dark gray, their corresponding mutant strains light
gray and other strains colored gray).

| | Compound MIC [μg/mL] | | | | | | | |
|---|---|---|---|---|---|---|---|---|
| # | Caspofungin | 1a | 1 | 2 | CSF-BOD | 5-TMR-azide | NBD-azide | BODIPY |
| 1. | 0.031 | 0.125 | 0.25 | 1 | 1 | >64 | >64 | >64 |
| 2. | 4 | 8 | 16 | 32 | 16 | >64 | >64 | >64 |
| 3. | 16 | 16 | 64 | >64 | >64 | >64 | >64 | >64 |
| 4. | 16 | 16 | 64 | 64 | >64 | >64 | >64 | >64 |
| 5. | 0.031 | 0.063 | 0.25 | 1 | 1 | >64 | >64 | >64 |
| 6. | 0.063 | 0.125 | 0.5 | 1 | 2 | >64 | >64 | >64 |
| 7. | 0.031 | 0.063 | 0.5 | 1 | 1 | >64 | >64 | >64 |
| 8. | 0.063 | 0.125 | 0.5 | 2 | 2 | >64 | >64 | >64 |
| 9. | 0.031 | 0.063 | 0.25 | 1 | 1 | >64 | >64 | >64 |
| 10. | 0.063 | 0.125 | 0.5 | 1 | 2 | >64 | >64 | >64 |
| 11. | 0.125 | 0.125 | 0.5 | 2 | 2 | >64 | >64 | >64 |
| 12. | 1 | 16 | 16 | 64 | 16 | >64 | >64 | >64 |
| 13. | 2 | 32 | 32 | >64 | 32 | >64 | >64 | >64 |
| 14. | 16 | 32 | 64 | >64 | >64 | >64 | >64 | >64 |
| 15. | >64 | >64 | >64 | >64 | >64 | >64 | >64 | >64 |
| 16. | 0.125 | 1 | 1 | 4 | 2 | >64 | >64 | >64 |
| 17. | 2 | 4 | 8 | 32 | 16 | >64 | >64 | >64 |
| 18. | 0.125 | 1 | 1 | 4 | 2 | >64 | >64 | >64 |
| 19. | >64 | >64 | >64 | >64 | >64 | >64 | >64 | >64 |
| 20. | >64 | >64 | >64 | >64 | >64 | >64 | >64 | >64 |
| 21. | 0.063 | 0.25 | 0.5 | 4 | 1 | >64 | >64 | >64 |
| 22. | >64 | >64 | >64 | >64 | >64 | >64 | >64 | >64 |
| 23. | >64 | >64 | >64 | >64 | >64 | >64 | >64 | >64 |
| 24. | 1 | 2 | 4 | 64 | 8 | >64 | >64 | >64 |
| 25. | >64 | >64 | >64 | >64 | >64 | >64 | >64 | >64 |
| 26. | 0.125 | 0.5 | 1 | 4 | 2 | >64 | >64 | >64 |
| 27. | >64 | >64 | >64 | >64 | >64 | >64 | >64 | >64 |
| 28. | 8 | >64 | >64 | >64 | >64 | >64 | >64 | >64 |
| 29. | 2 | >64 | >64 | >64 | >64 | >64 | >64 | >64 |
| 30. | >64 | >64 | >64 | >64 | >64 | >64 | >64 | >64 |
| 31. | 0.063 | 0.5 | 0.5 | 2 | 1 | >64 | >64 | >64 |
| 32. | 8 | 16 | 64 | >64 | >64 | >64 | >64 | >64 |
| 33. | >64 | >64 | >64 | >64 | >64 | >64 | >64 | >64 |
| 34. | 0.125 | 0.5 | 1 | 4 | 2 | >64 | >64 | >64 |
| 35. | >64 | >64 | >64 | >64 | >64 | >64 | >64 | >64 |
| 36. | >64 | >64 | >64 | >64 | >64 | >64 | >64 | >64 |
| 37. | >64 | >64 | >64 | >64 | >64 | >64 | >64 | >64 |
| 38. | 16 | >64 | >64 | >64 | >64 | >64 | >64 | >64 |
| 39. | 0.125 | 0.5 | 1 | 2 | 1 | >64 | >64 | >64 |
| 40. | 0.125 | 1 | 1 | 2 | 2 | >64 | >64 | >64 |
| 41. | 0.125 | 1 | 1 | 4 | 2 | >64 | >64 | >64 |
| 42. | 0.063 | 0.5 | 0.5 | 2 | 1 | >64 | >64 | >64 |
| 43. | 0.063 | 0.25 | 0.5 | 2 | 1 | >64 | >64 | >64 |
| 44. | 0.063 | 0.5 | 0.5 | 2 | 2 | >64 | >64 | >64 |
| 45. | 0.125 | 1 | 1 | 4 | 1 | >64 | >64 | >64 |
| 46. | 0.063 | 0.25 | 0.5 | 2 | 1 | >64 | >64 | >64 |
| 47. | 0.063 | 0.25 | 0.5 | 2 | 2 | >64 | >64 | >64 |
| 48. | 0.063 | 0.5 | 0.5 | 2 | 2 | >64 | >64 | >64 |
| 49. | 0.125 | 0.5 | 1 | 4 | 2 | >64 | >64 | >64 |

Based on MIC values for compounds 1a, 1 and 2, all three caspofungin derivatives retained antifungal activities. The MIC values of 1a, 1 and 2, were 1-8, 4-16 and 16-16 higher than the parent caspofungin, respectively (FIG. 2, Table 2). MIC values of CSF-BOD were 8-32-fold higher than those of caspofungin against the responsive strains from the tested panel. Given that the molecular weight and size of TMR dye is over 2-fold higher than that of NBD, it was surprising to find that the MIC values of probe 2 (the NBD-labeled caspofungin) were higher than those of probe 1 (the TMR-labeled caspofungin). Furthermore, this difference was more evident for C. albicans strains than for the C. glabrata strains (FIG. 2). NBD is a non-charged fluorescent dye, but TMR is zwitterionic. This feature can modulate the binding interactions between the fluorescent probe and its target site in the GS complex and may account for the antifungal efficacy differences between probes 1 and 2. Importantly, the antifungal activity spectrum of the three fluorescent probes was identical to that of caspofungin: caspofungin-resistant strains were also resistant to CSF-BOD, compound 1 and compound 2, and caspofungin responsive strains were also sensitive to the probes. This is consistent with the idea that caspofungin and its fluorescent probes share the same mode of action.

### Example 3. Fluorescent Caspofungin Probes Accumulate in Yeast Cells Vacuole Via Endocytosis

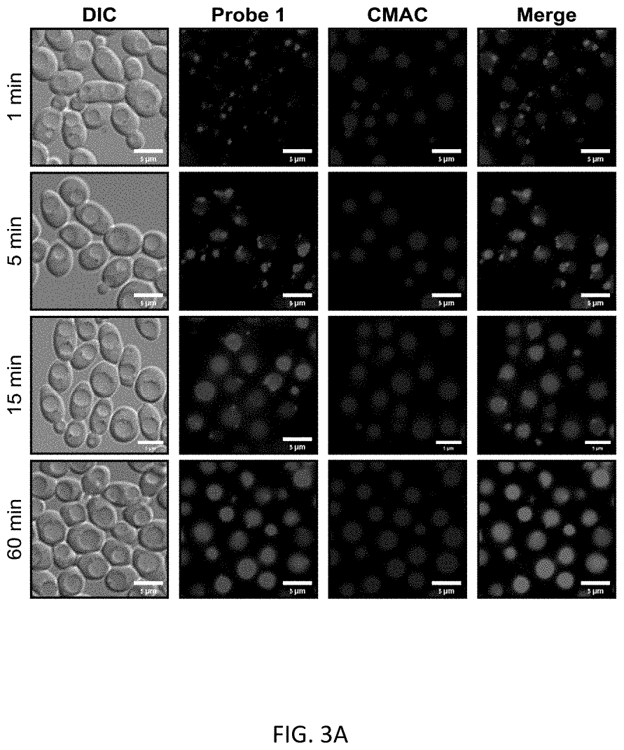

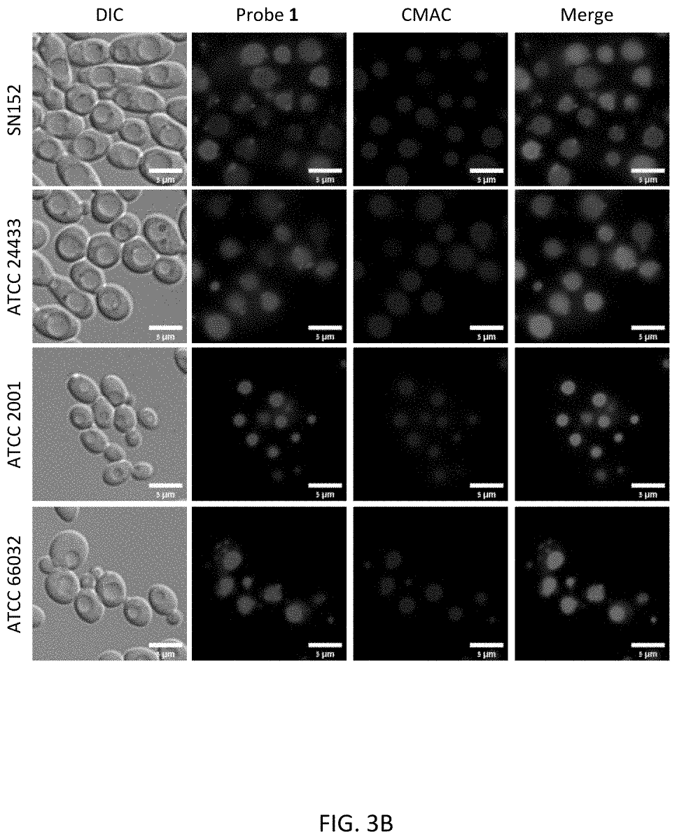

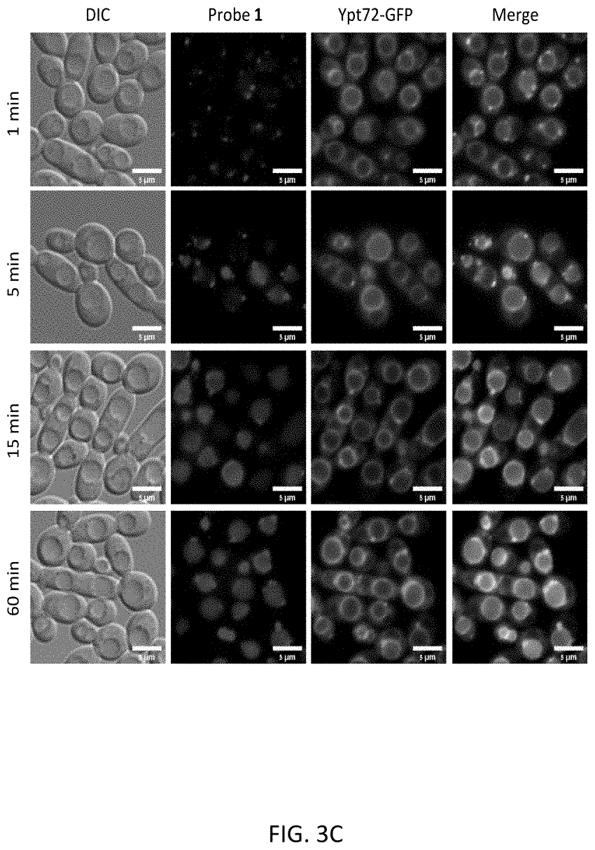

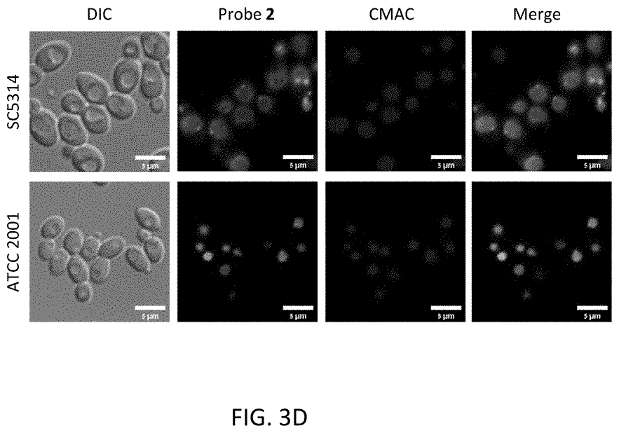

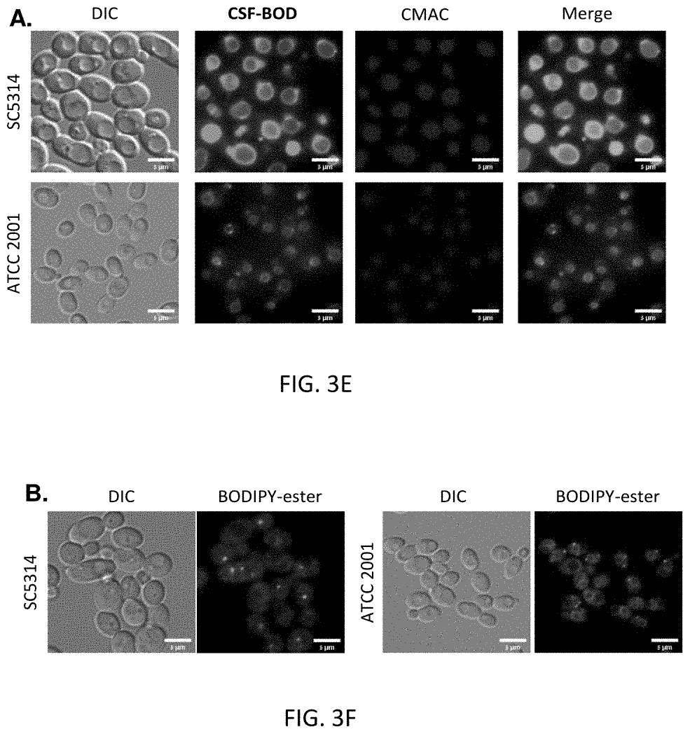

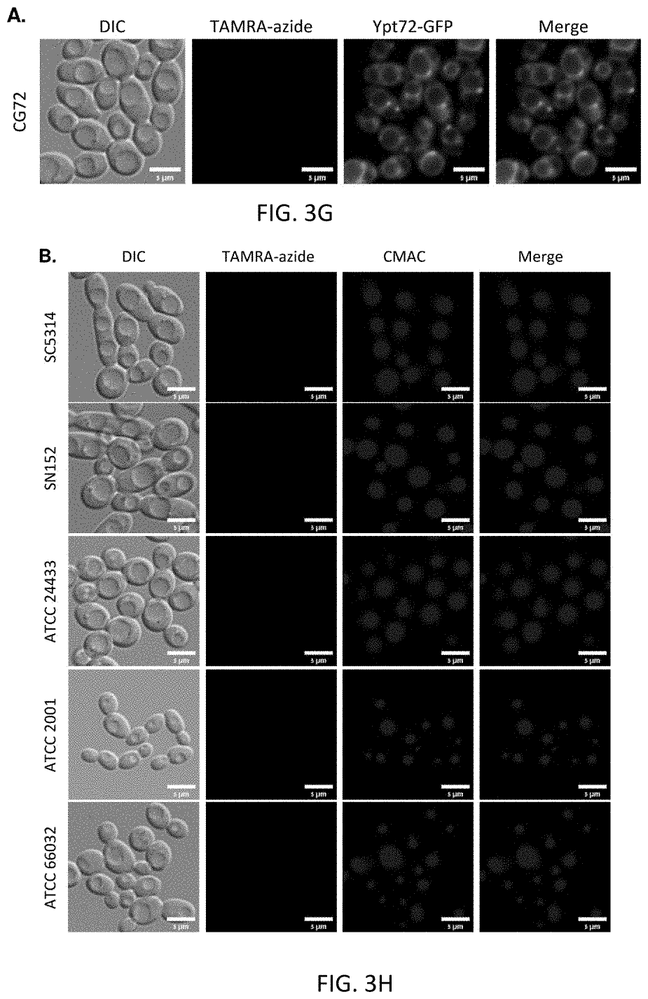

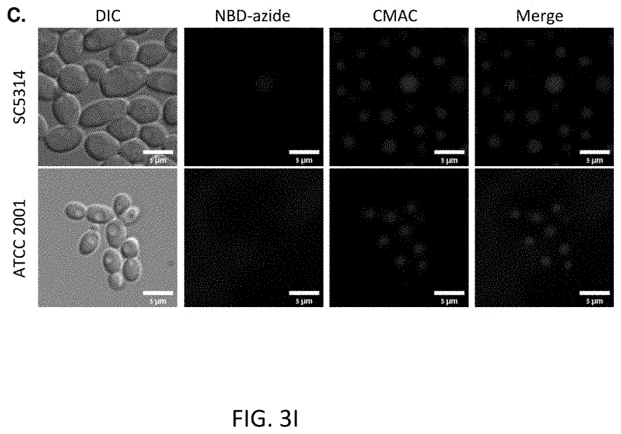

The cellular distribution of the fluorescent caspofungin probes was determined for four C. albicans and two C. glabrata strains using live cell fluorescence microscopy. Within 15 min, probe 1 localized to the vacuole, as determined by its co-localization with the vacuole-specific fluorescent dye CellTracker™ Blue 7-amino-4-chloromethylcoumarin (CMAC) (FIG. 3A). To learn about how the fluorescent echinocandin transited into the vacuole, a time-lapse analysis was performed which revealed fluorescently labeled vesicular structures, reminiscent of endosomes, within 1 min after the addition of probe 1 (1 μM; FIG. 3A, 1 min). These vesicles first accumulated in close proximity to, and then within, the vacuole (FIG. 3A, 5 min), ultimately labeling the entire vacuole within ˜15 minutes (FIG. 3A, 15 min). The probes remained concentrated at the vacuole for 60 min (FIG. 3, 60 min). Importantly, the same localization pattern was evident for both TMR-based probe 1 and NBD-based probe 2 and for CSF-BOD (FIGS. 3B-3F) in all of the strains tested; no such staining was observed in yeast cells treated with the free azide functionalized TMR or NBD dyes used for the preparation of probe 1 or probe 2 (FIGS. 3G-3I). Thus, the echinocandin scaffold, and not the conjugated fluorescent dye moieties, conferred the subcellular distribution of the two fluorescent probes to endocytic vesicles that subsequently fused with the vacuole.

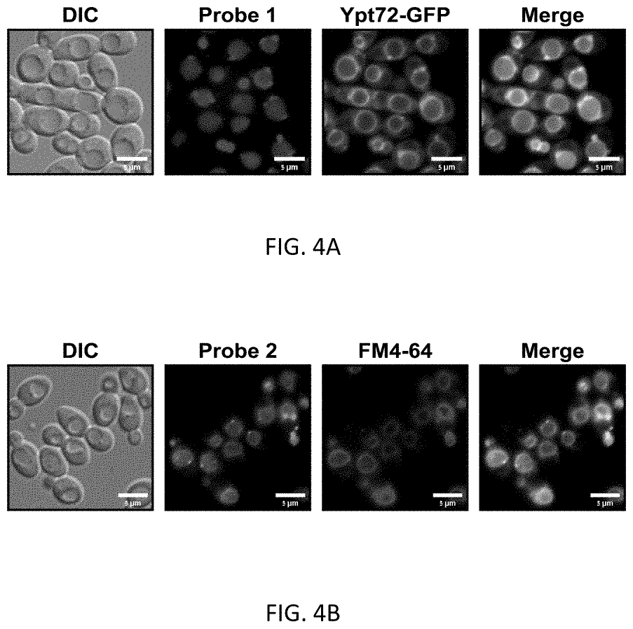

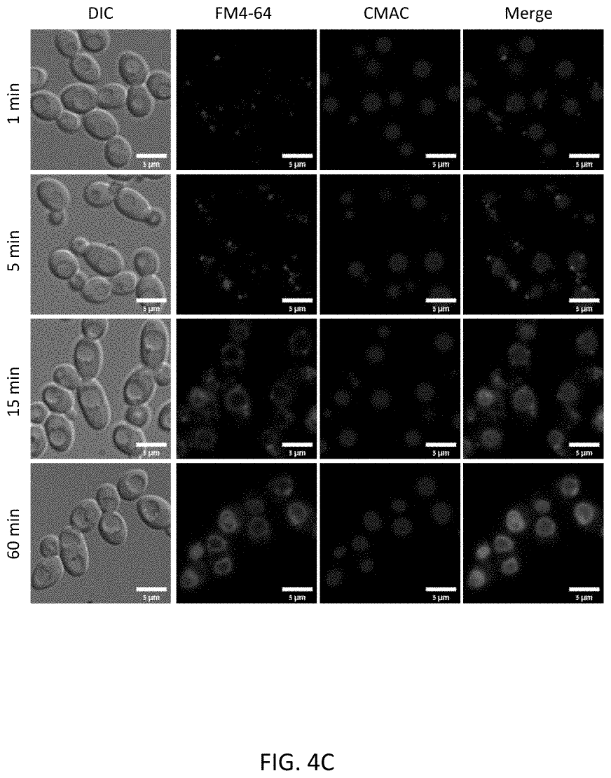

If the caspofungin probes are internalized via endocytosis, then in C. albicans, the intracellular probes should be surrounded by Ypt72, a vacuolar Rab small monomeric GTPase orthologous with S. cerevisiae Ypt7, that localizes primarily to the vacuolar membrane. Additionally, the fluorescent caspofungin probe should form a fluorescent pattern of endocytic vesicles similar to that observed in yeast stained with FM4-64, a fluorescent dye that localizes to endocytic vesicles and the vacuolar membrane. Indeed, when probe 1 was used with C. albicans cells that express Ypt72-GFP, (strain CG72, Table 1), the GFP labeled vacuolar membrane surrounded probe 1 (FIG. 4A, and FIG. 3C).

A similar pattern was seen with probe 2 relative to FM4-64 (FIG. 4B), further supporting the idea that the internalization of either of the fluorescent caspofungin probes occurs via endocytosis in C. albicans. A similar endosomal pattern to that observed in cells stained with FM4-64, was seen when cells were incubated with probe 1 for a short period of time (FIG. 4C, 1-5 min.), further supporting the idea that internalization of either of the fluorescent caspofungin probes into Candida cells occurs via endocytosis

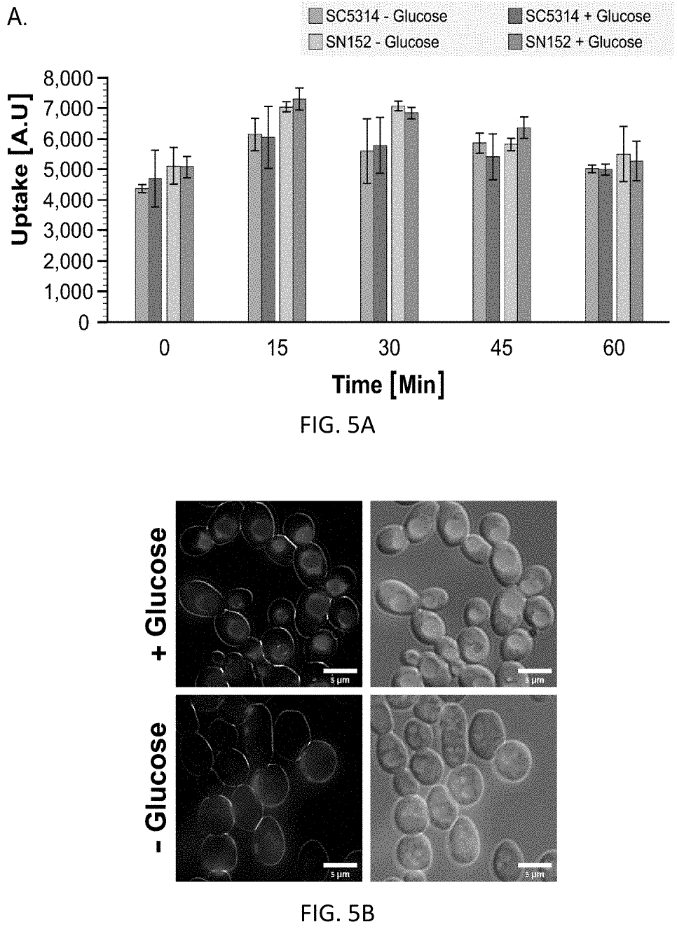

Endocytosis is an energy-dependent process. To determine whether the intracellular uptake of fluorescent caspofungin is energy-dependent, the effect of glucose on the uptake of probe 1 was evaluated. Cells from common C. albicans yeast laboratory strains (SC5314 and SN152, respectively, Table 1) were suspended in PBS with or without 2% glucose. After 4 hours of incubation, probe 1 was added to a final concentration of 1 μM and its cellular uptake was measured by flow cytometry every 15 min for 1 hour (FIG. 5A). The differences in the uptake of probe 1 in the glucose-rich PBS solution, relative to the glucose-free PBS, during the 60 min of the experiment were not significant (FIG. 5A), suggesting that the uptake of probe 1 might not be energy dependent.

In the presence of glucose, probe 1 readily crossed the membrane and accumulated in the vacuole (FIG. 5B). By contrast, accumulation in the vacuole was clearly energy-dependent: in the absence of glucose, probe 1 accumulated primarily at the cell envelope of yeast cells (FIG. 5B). These results support the idea that intracellular uptake of echinocandins is dependent upon endocytosis, which is an energy-dependent process. Furthermore, the fluorescence microscopy results can explain the apparent paradox between the flow cytometry results, which measure fluorescence per cell, irrespective of its localization within the cell and the microscopy results: the degree of probe 1 staining at the cell surface in the absence of glucose and its staining of the vacuole were of the same magnitude (t test: P-value >0.05; Table 3), which represents analysis, by microscopy (as in FIG. 5B), with or without 2% glucose, presented as integrated density (RFU=relative fluorescent units) per cell (determined from microscopic images with ImageJ). Thus, the drug associates with the cell surface in an energy independent manner, but its uptake in vacuole-directed vesicles requires energy.

TABLE 3
Quantification of fluorescence intensity of
probe 1 in cells

| Glucose | Integrated density per cell (RFU) |
|---|---|
| + | (8.29 ± 2.56) × 10⁶ |
| − | (7.99 ± 1.64) × 10⁶ |

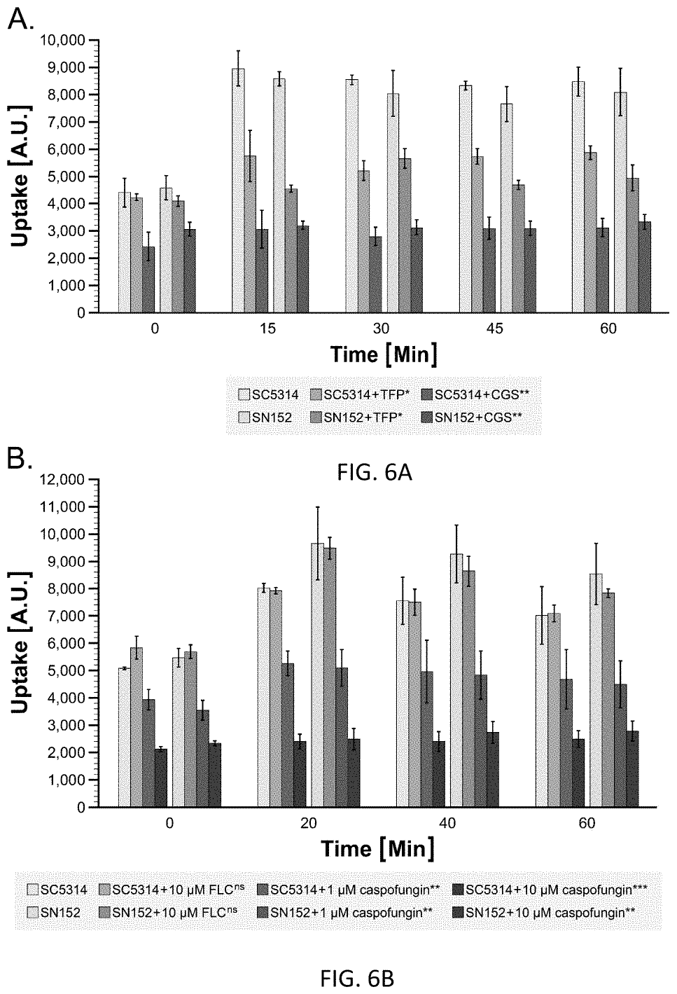

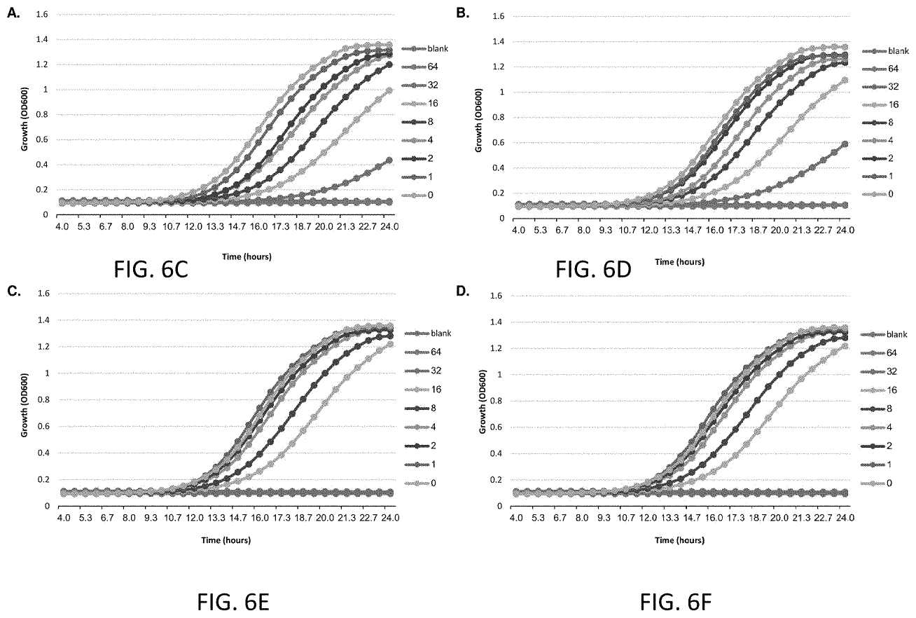

Assuming that echinocandin probe uptake occurs via endocytosis, then inhibitors of endocytosis should inhibit probe uptake as well. The accumulation of probe 1 in C. albicans SC5314 and SN152 cells was measured by flow cytometry in the absence and presence of endocytosis inhibitors trifluoperazine (TFP) or pyrroloquinoxaline derivative CGS 12066B (FIG. 6A). TFP is a dopamine receptor antagonist and CGS 12066B is a serotonin-1B receptor agonist, both are known to modestly reduce growth at the concentration used (8 μg/mL): they primarily slowed down the emergence from lag phase (FIGS. 6C-6F).

Notably, both endocytosis inhibitors significantly decreased the uptake of probe 1 relative to uptake levels in untreated yeast cells, with the effect of CGS 12066B being more pronounced than that of TFP (FIG. 6A). For example, after 30 min, TFP inhibited the uptake of probe 1 by approximately 30% and CGS 12066B inhibited its uptake by approximately 60% in both C. albicans SC5314 and SN152. These results, together with live cell imaging and glucose starvation data, strongly support the idea that intracellular uptake of fluorescent caspofungin probe 1 occurs rapidly, within minutes, largely via endocytic vesicles that drive vacuolar accumulation. occurs, within minutes, largely via endocytic vesicles that drive vacuolar accumulation of the antifungal drug.

To determine if caspofungin competes for cellular uptake with fluorescent probe 1, both the drug and the probe were added simultaneously to cultures of strains SC5314 and SN152. The uptake of probe 1 was measured by flow cytometry every 15 minutes for 1 hour (FIG. 6B). Addition of caspofungin reduced the uptake of probe 1 in a dose-dependent manner. No effect on the uptake of probe 1 was detected in cells co-treated with the antifungal azole drug fluconazole, which inhibits the biosynthesis of fungal membrane sterol, ergosterol. These results suggest that the endocytic internalization of echinocandins into yeast cells is mediated by binding of the drug to a membrane protein target, possibly the GS complex. Remarkably, in the important fungal pathogen Aspergillus fumigatus, caspofungin was shown to induce dynamic changes in the localization of Fks1p from the membrane to the vacuole. This study suggests that endocytic migration of Fks1p from the plasma membrane to the vacuole may be involved, at least in part, in vacuolar accumulation of echinocandins.

### Example 4. Echinocandins are More Effective Against Dividing than Quiescent Cells

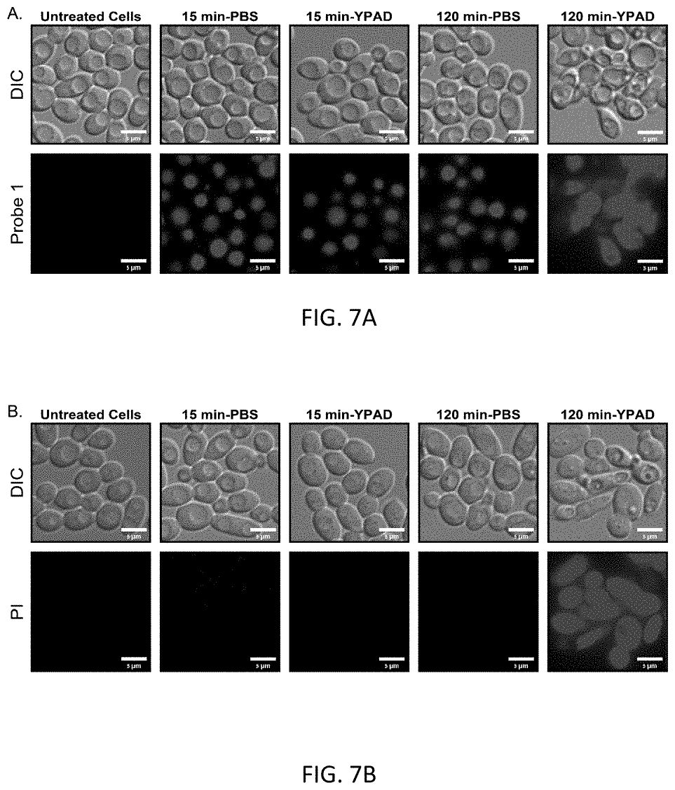

The localization of probe 1 was very different in Candida cells incubated for 2 hours in nutrient rich medium, Yeast Extract Peptone Dextrose medium plus Adenine (YPAD), or in nutrient-free PBS buffer (FIG. 7A). For cells in PBS alone, the probe remained concentrated at the vacuole over the 2-hour duration of the experiment (FIG. 7A). By contrast, for cells in YPAD, after 2 h, vacuolar pattern of the caspofungin probe had dispersed, and the entire cytoplasm was brightly stained (FIG. 7A). DIC imaging of probe 1 in cells grown in YPAD revealed larger, more misshapen cells that often appeared to be collapsed, a sign of yeast cell death. Thus, the caspofungin probe was relocalized from the vacuole, causing more cell damage, and possibly cell death, in cells incubated in nutrient-rich growth medium, while cells in PBS retained probe 1 in the vacuole and did not appear to be undergoing cell damage.

To determine whether the caspofungin relocalization and apparent cell damage and death is a feature of caspofungin (and not only of the fluorescent caspofungin probe 1), cell viability was followed using propidium iodide (PI), a dye normally excluded from metabolically active cells.

Cell viability was compared in the presence and absence of 1 μM unlabeled caspofungin, using cells incubated either in PBS or in YPAD. In PBS, cells excluded the PI stain (FIG. 7B), indicating that the plasma membrane remained intact and the cells were viable. By contrast, in YPAD medium, cells were viable after 15 min, but stained brightly with PI after 2 h (FIG. 7B). Thus, cells exposed to caspofungin in nutrient-rich conditions become permeable to PI, an indicator of cell death. This observation explains the non-specific distribution of probe 1 in the cytoplasm after prolonged incubation (FIG. 7A). It is also consistent with the idea that plasma membrane permeabilization and cell death are likely accompanied by the loss of structural integrity of organelles, including the vacuoles detected here.

There are many differences between YPAD and PBS media, the most general of which is YPAD contains nutrients that promote cell growth while PBS simply maintains cells in a quiescent state. Cell growth is dependent upon cell-wall expansion, which in turn requires β-glucan production by GS. Hence, without being bound by any theory or mechanism, it is proposed that growing cells are more sensitive to caspofungin because if cell-wall integrity is compromised, growth will lead to cell deformation, membrane rupture and cell death. By contrast, if cells are held in a quiescent environment that maintains viability but does not stimulate growth and cell cycle progression, the drug can be taken up into the vacuole; in this case, despite the lack of β-glucan synthesis, the cells can continue to survive.

### Example 5. Enhanced Uptake of Probe 1 in Caspofungin-Resistant Candida Strains

Several isogenic sets of parent/progeny strains in which the progeny strains are caspofungin-resistant due to point mutations in the FKS genes, were exploited. These included four C. albicans strains (Strains 1-4, Table 1), and a collection of 23 C. glabrata strains in 6 different genetic backgrounds (Strains 16-38, Table 1).

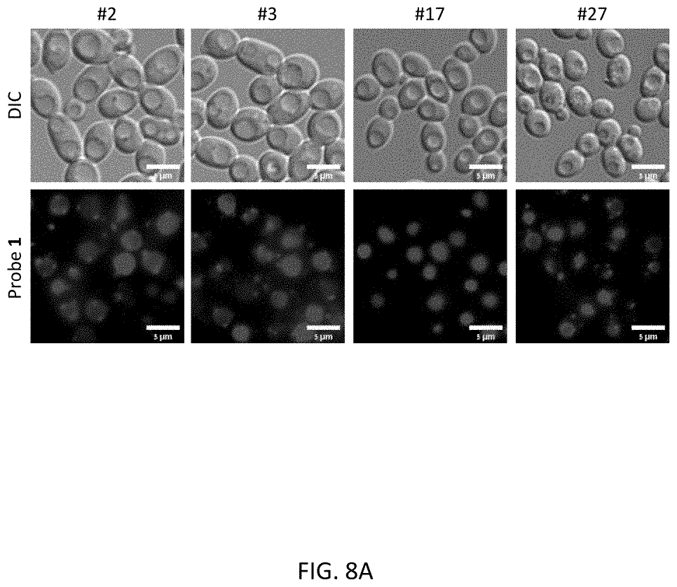

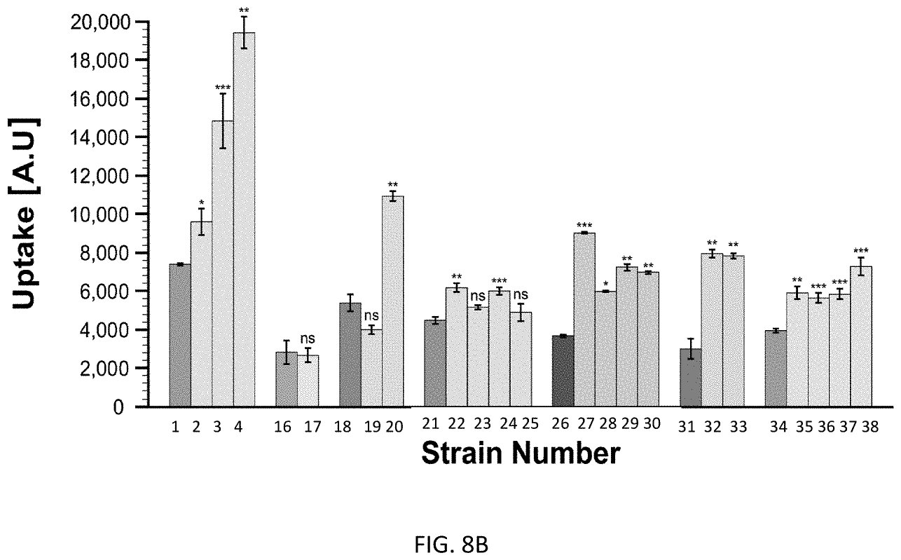

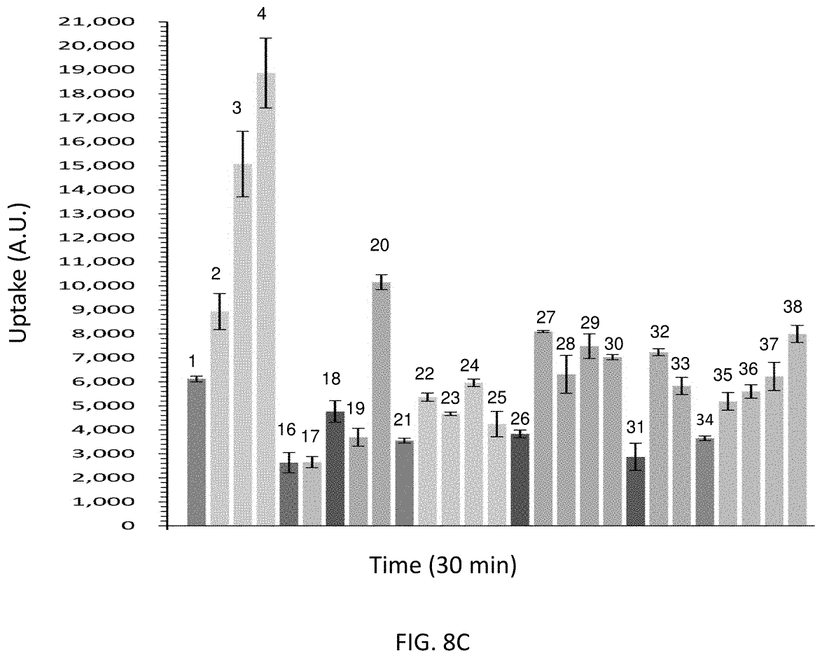

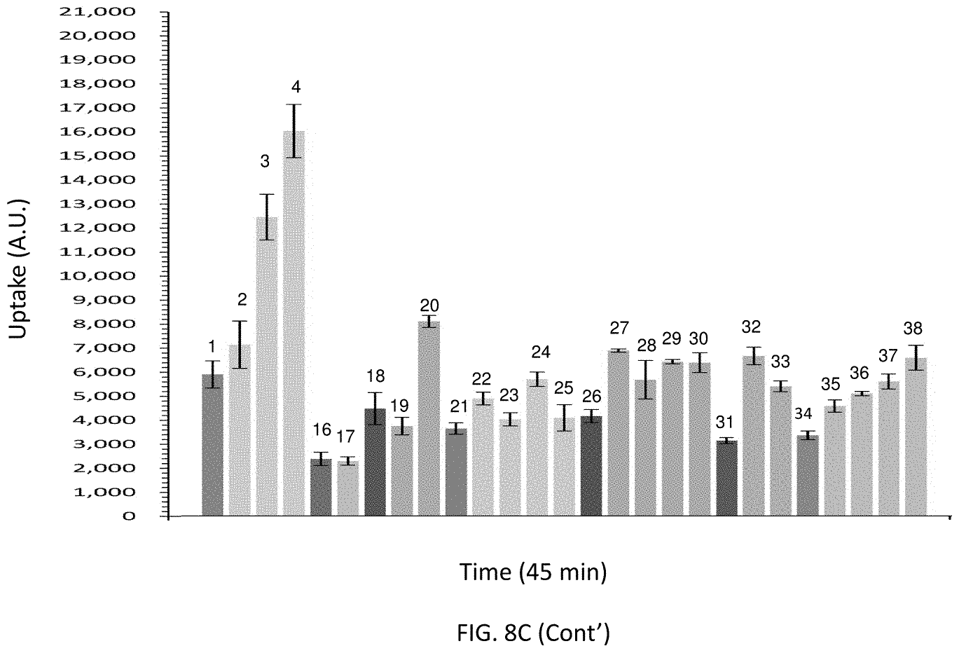

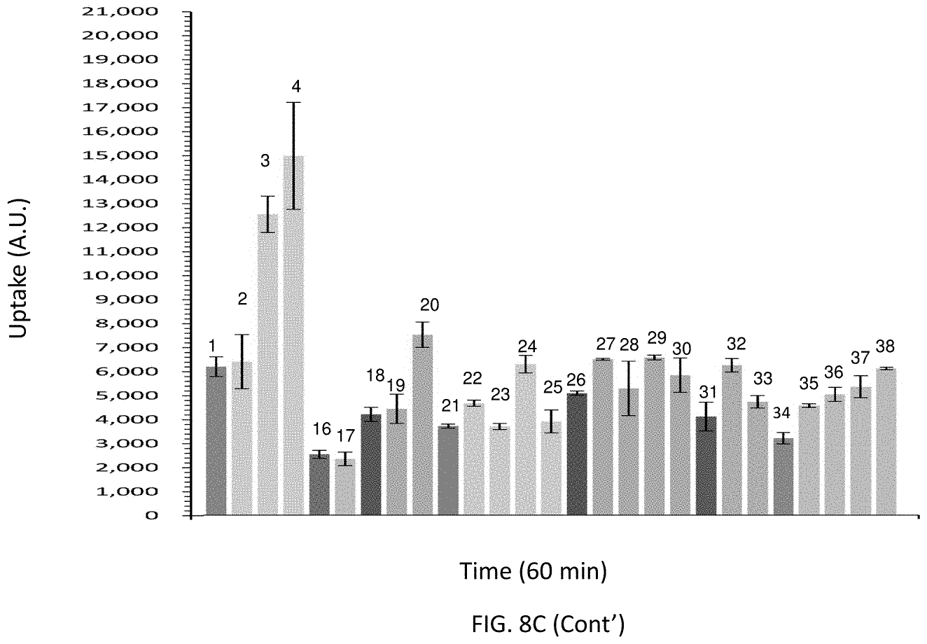

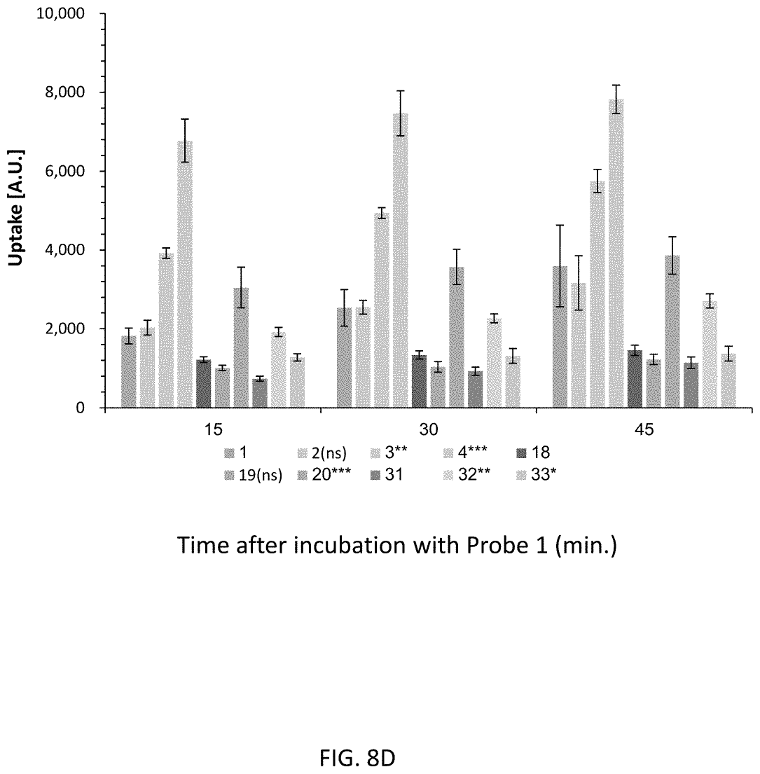

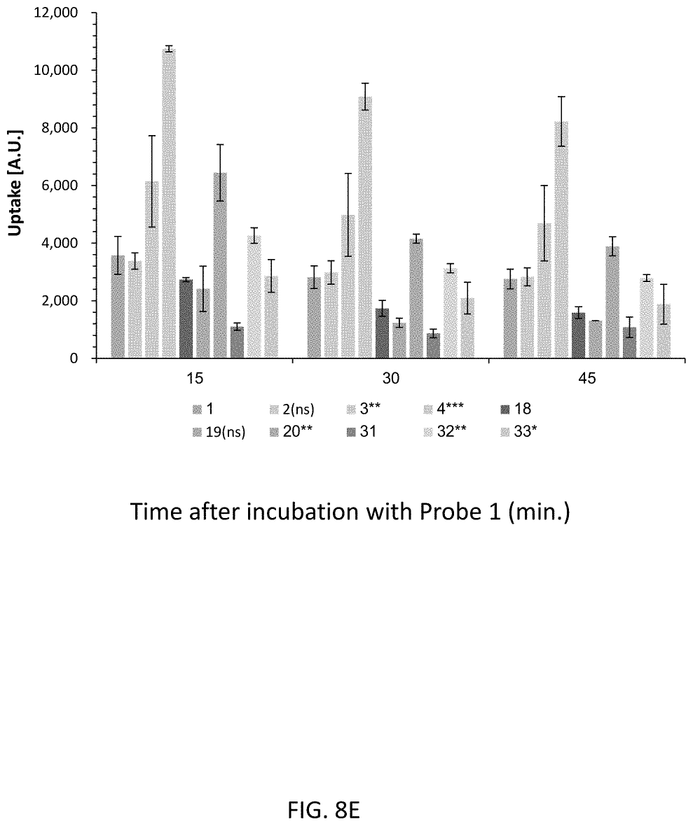

Initially, subcellular localization of caspofungin probe 1 in four echinocandin-resistant progeny strains from the panel was determined by microscopy (FIG. 8A). It has been found that the probe localized to the vacuole as it did in echinocandin-responsive strains. Thereafter, the dynamics of cellular association with probe 1 was quantified by flow cytometry at 15-minute intervals for one hour (FIGS. 8B-8C). A statistically significant increase in probe 1 uptake was observed in 80% of the resistant strains relative to the corresponding parental strains (FIG. 8B). Since all of the caspofungin-resistant strains in the panel are mutants in one of the two FKS genes that encode GS (Table 1), these results suggest a possible connection between FKS mutations and the uptake of probe 1.

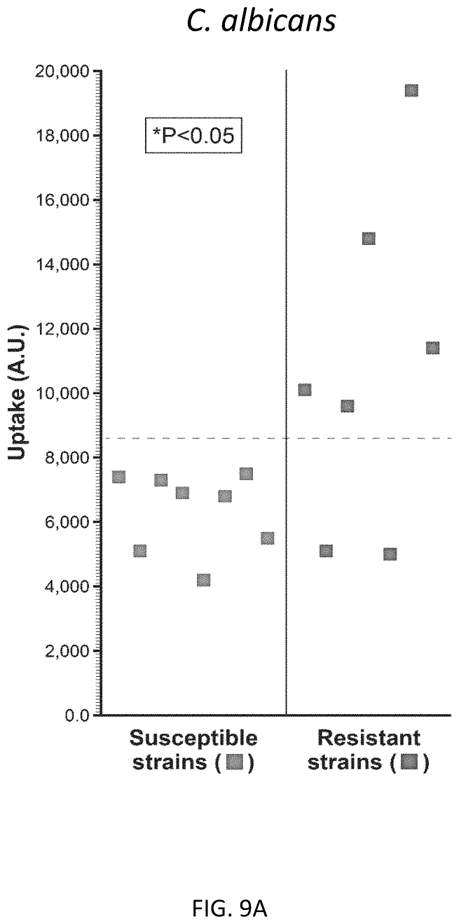

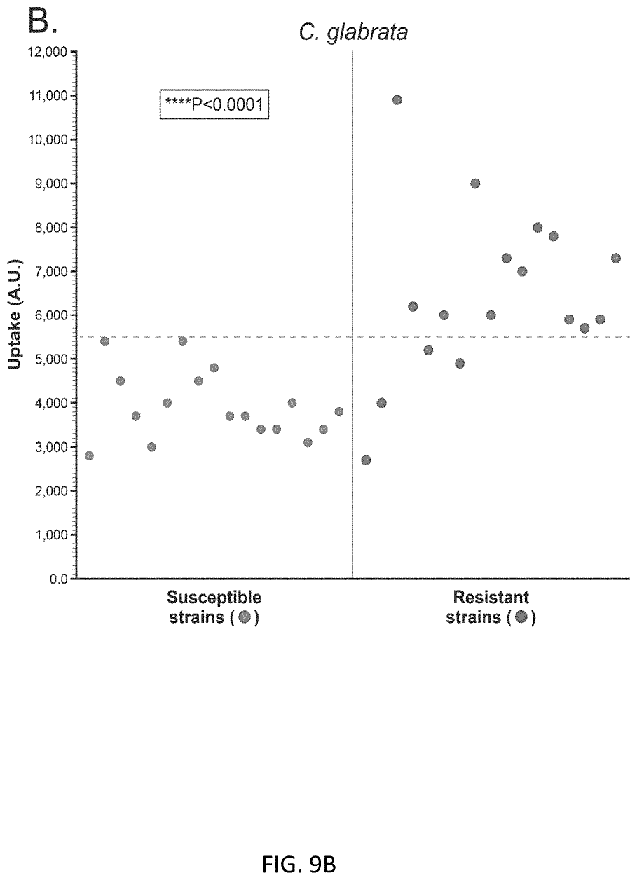

To determine whether increased uptake by echinocandin-resistant strains is a more general phenomenon that spans most genetic backgrounds, a set of C. albicans clinical isolates that span a broad range of sensitivity/resistance (Strains 5-15, Table 1) were scanned, as well as a set of C. glabrata responsive clinical isolates (Strains 39-49, Table 1) in addition to the strains in FIG. 8B. Probe 1 uptake for caspofungin-resistant strains was significantly higher than for caspofungin-responsive strains (P-value <0.05 and <0.0001 for C. albicans and C. glabrata, respectively) (FIGS. 9A and 9B). The caspofungin-responsive C. albicans and C. glabrata strains had average levels of fluorescence of 6.3±0.5 and 3.9±0.2, respectively, and the caspofungin-resistant strains were 10.8±1.4 and 6.5±0.5, respectively. Importantly, 71% of C. albicans and 77% of the C. glabrata echinocandin-resistant strains tested resistant using the calculated cutoff lines from the mutant strains (FIGS. 9A and 9B). Indeed, all (100%) of strains with uptake values above the cutoff lines were confirmed as resistant. Thus, on average, increased uptake of fluorescent caspofungin probe 1 appears to be associated with echinocandin resistance. The NBD- and BODIPY-labeled caspofungin probes behaved similarly to the TMR-based probe (FIG. 8D-8E) in resistant strains relative to the corresponding parental strains. Without being bound by any theory or mechanism of action, this observation may be due to the fact that the caspofungin scaffold, and not the fluorescent dye or the labeling position on the drug, is responsible for the observed enhanced uptake of the fluorescent probes in echinocandin resistant strains. Given that the detection process requires only minutes (less than 30 minutes) to complete, the assay disclosed herein provides a rapid and useful detection tool for identifying echinocandin-resistant isolates of pathogenic yeast.

### Example 6. Elevated Cell-Wall Chitin and Caspofungin Probe 1 Uptake

The fungal cell-wall is a dynamic matrix, and a decrease in one of its components is usually compensated by an increase in another. It is well-established that caspofungin-mediated inhibition of β-glucan synthase results in increased cell-wall chitin production in Candida. Elevated cell-wall chitin levels are also detected in FKS mutants of C. albicans that confer echinocandin resistance. Thus, it is unclear whether elevated levels of probe 1 uptake correlate with elevated levels of cell-wall chitin in echinocandin-resistant Candida isolate.

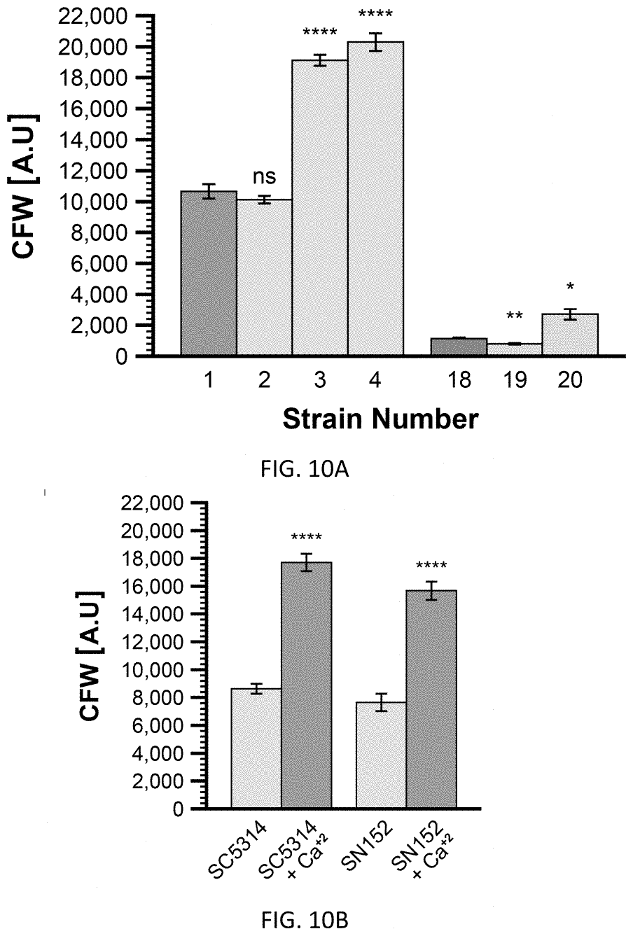

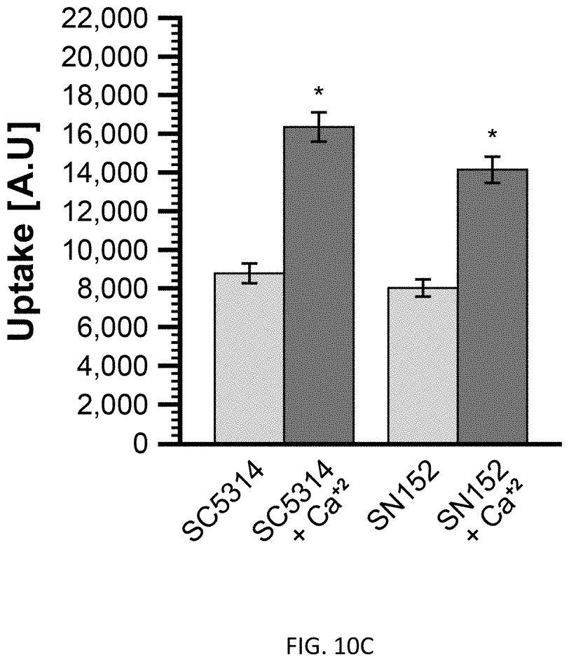

First chitin levels were measured in caspofungin-responsive C. albicans SC5314 and three of its caspofungin-resistant progeny strains (Strains 1-4, Table 1) as well as in caspofungin-responsive C. glabrata CST109 and two of its caspofungin-resistant progeny strains (Strains 18-20, Table 1). Chitin levels were measured by flow cytometry as the fluorescent signal in cell samples that were stained with the chitin-specific dye calcofluor white (CFW). A significant elevation in chitin levels was measured in cells of the caspofungin-resistant C. albicans progeny strains relative to their responsive parent, with the exception of strain 2 (FIG. 10A). Similarly, a significant increase in chitin was detected in C. glabrata cells of the progeny strain 20, but not of progeny strain 19. Importantly, the chitin levels in the strains correlated well with the uptake of fluorescent caspofungin probe 1. Indeed, the two strains (2 and 19) that did not show elevated chitin staining (FIG. 10A), also showed very little difference in their probe 1 uptake relative to their parent strains (FIG. 8B).

Ca²⁺ is known to enhance the chitin content of cell-walls and to reduce sensitivity to caspofungin susceptibility. To determine the relationship between sensitivity to caspofungin, chitin production and the uptake of fluorescent caspofungin probe 1, caspofungin responsive C. albicans cells (SC5314 and SN152) were pre-incubated with 0.1M of Ca²⁺ for 18 hours to stimulate elevation in of chitin content in the cell-wall and then were stained with CFW or with 1 and analyzed by flow cytometry. We detected an approximately 2-fold elevation in chitin level (FIG. 10B) along with an approximately 2-fold elevation in the uptake of probe 1 when cells were supplemented with Ca²⁺ relative to yeast cells that were not supplemented with Ca²⁺. Of note, in C. albicans cells stained with CFW or with probe 1, similar fluorescent signal intensities were measured. By contrast, in C. glabrata, the signal intensity of CFW was significantly lower than that of probe 1. Thus, in C. glabrata, CFW appears to be a less sensitive indicator of caspofungin-resistance than probe 1 uptake (compare strains 18-20 in FIG. 10A and FIG. 8B). It is known that the percentage of chitin in cell wall of C. glabrata is significantly lower than that in C. albicans. This likely explains the observation that in C. glabrata, the chitin stain CFW is a less sensitive indicator of caspofungin-resistance.

In addition, these results suggest that increasing chitin levels in the cell-wall results in increased uptake of echinocandins into vacuoles via endocytosis. Chitin enhances cell-wall rigidity and the ability of the cell-wall to counter intracellular turgor pressure that can affect the equilibrium between endocytic and exocytic processes. This mechanism suggests a plausible explanation for the observed elevation in endocytic vacuolar uptake of fluorescent caspofungin probe 1 in echinocandin-resistant yeast that have higher levels of cell-wall chitin.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1-30. (canceled)

31. A caspofungin derivative comprising a modified phenol wherein the caspofungin derivative has anti-fungal activity.

32. The caspofungin derivative according to claim 31, wherein the modified phenol comprises an azide moiety or a propargyl group.

33. The caspofungin derivative according to claim 32, wherein the azide moiety comprises 3-azide propylamine.

34. The caspofungin derivative according to claim 31, selected from the group consisting of: 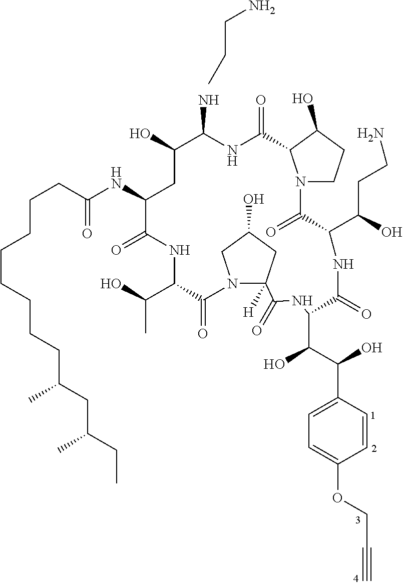
Compound 1a; 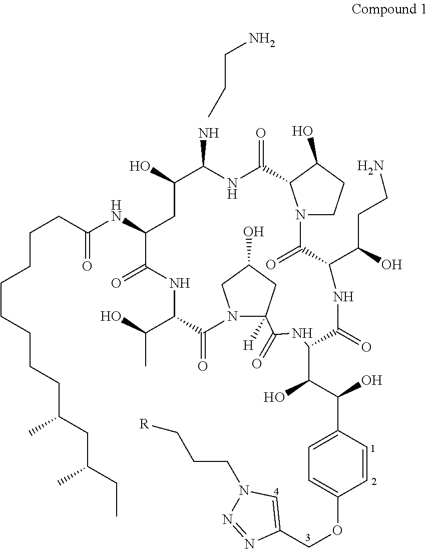
wherein R is: 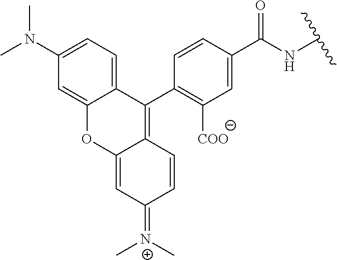
and 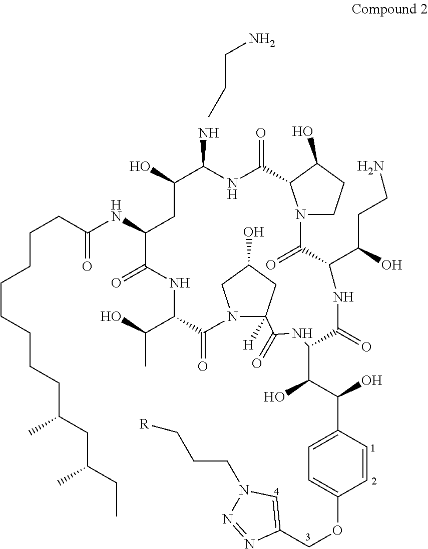
wherein R is:

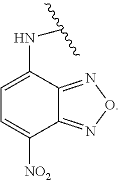 [SMILES: *Nc1ccc([N+](=O)[O-])c2nonc12]

35. A pharmaceutical composition comprising the caspofungin derivative of claim 31, and a pharmaceutically acceptable excipient.

36. A method of treating fungal infection in a subject in need thereof, the method comprising administering to the subject in need thereof the pharmaceutical composition according to claim 35.

37. The method according to claim 36, wherein said administering comprises topical administration.

38. A method for determining responsiveness to an anti-fungal activity of an echinocandin compound, the method comprising contacting a yeast cell with an echinocandin compound and determining vacuolar uptake of the echinocandin compound.

39. The method according to claim 38, wherein vacuolar uptake below threshold indicates that said yeast cell is responsive to the antifungal activity of said echinocandin compound.

40. The method according to claim 38, wherein the echinocandin compound comprises a detectable label, and wherein said determining comprises detecting the level of the detectable label.

41. The method of claim 40, wherein the detectable label comprises fluorophores.

42. The method according to claim 38, wherein the echinocandin compound comprises caspofungin or caspofungin derivative.

43. The method according to claim 38, wherein vacuolar uptake above threshold indicates that said yeast cell is resistant to the antifungal activity of said echinocandin compound.

44. A method of treating fungal infection in a subject in need thereof, the method comprising
a. obtaining a sample from a tissue derived from the subject in need thereof;
b. contacting the sample with an echinocandin compound;
c. determining the vacuolar uptake of the echinocandin compound; and
d. administering to the subject in need thereof a pharmaceutical composition comprising the echinocandin compound when the vacuolar uptake of the echinocandin compound is below a threshold.

45. The method according to claim 44, wherein the echinocandin compound comprises caspofungin or a caspofungin derivative.

46. The method according to claim 44, wherein the echinocandin compound comprises caspofungin.

47. The method according to claim 45, wherein the caspofungin derivative is selected from Compound 1, Compound 1a and Compound 2.

48. The method according to claim 44, wherein the echinocandin compound comprises a detectable label.

49. The method according to claim 44, wherein the tissue is selected from: mucus secretion, blood, saliva, urine, plasma and epithelial tissue.

50. The method according to claim 44, wherein said administering comprises topical administration.
